SEQUENCE LISTING (12 sequences)

SEQ ID NO: 1 - DNA, 1002 bp, Homo sapiens
aaccggctttccgtatttttttcagtaatcttcatgacagtgggaatcttgtcaaacagccttgccatcgccattctcatgaaggcatatcagagatttagacagaagtccaaggcatcgtttctgcttttggccagtggcctggtaatcactgatttctttggccatctcatcaatggagccatagcagtatttgtatatgcttctgataaagaatggatccgctttgaccaatcaaatgtcctttgcagtatttttggtatctgcatggtgttttctggtctgtgcccacttcttctaggcagtgtgatggccattgagcggtgtattggagtcacaaaaccaatatttcattctacgaaaattacatccaaacatgtgaaaatgatgttaagtggtgtgtgcttgtttgctgttttcatagctttgctgcccatccttggacatcgagactataaaattcaggcgtcgaggacctggtgtttctacaacacagaagacatcaaagactgggaagatagattttatcttctacttttttcttttctggggctcttagcccttggtgtttcattgttgtgcaatgcaatcacaggaattacacttttaagagttaaatttaaaagtcagcagcacagacaaggcagatctcatcatttggaaatggtaatccagctcctggcgataatgtgtgtctcctgtatttgttggagcccatttctggttacaatggccaacattggaataaatggaaatcattctctggaaacctgtgaaacaacactttttgctctccgaatggcaacatggaatcaaatcttagatccttgggtatatattcttctacgaaaggctgtccttaagaatctctataagcttgccagtcaatgctgtggagtgcatgtcatcagcttacatatttgggagcttagttccattaaaaattccttaaaggttgctgctatttctgagtcaccagttgcagagaaatcagcaagcacc

SEQ ID NO: 2 - protein, 334 aa, Homo sapiens
NRLSVFFSVIFMTVGILSNSLAIAILMKAYQRFRQKSKASFLLLASGLVITDFFGHLINGAIAVFVYASDKEWIRFDQSNVLCSIFGICMVFSGLCPLLLGSVMAIERCIGVTKPIFHSTKITSKHVKMMLSGVCLFAVFIALLPILGHRDYKIQASRTWCFYNTEDIKDWEDRFYLLLFSFLGLLALGVSLLCNAITGITLLRVKFKSQQHRQGRSHHLEMVIQLLAIMCVSCICWSPFLVTMANIGINGNHSLETCETTLFALRMATWNQILDPWVYILLRKAVLKNLYKLASQCCGVHVISLHIWELSSIKNSLKVAAISESPVAEKSAST

SEQ ID NO: 3 - DNA, 816 bp, Homo sapiens
aaccggctttccgtatttttttcagtaatcttcatgacagtgggaatcttgtcaaacagccttgccatcgccattctcatgaaggcatatcagagatttagacagaagtccaaggcatcgtttctgcttttggccagtggcctggtaatcactgatttctttggccatctcatcaatggagccatagcagtatttgtatatgcttctgataaagaatggatccgctttgaccaatcaaatgtcctttgcagtatttttggtatctgcatggtgttttctggtctgtgcccacttcttctaggcagtgtgatggccattgagcggtgtattggagtcacaaaaccaatatttcattctacgaaaattacatccaaacatgtgaaaatgatgttaagtggtgtgtgcttgtttgctgttttcatagctttgctgcccatccttggacatcgagactataaaattcaggcgtcgaggacctggtgtttctacaacacagaagacatcaaagactgggaagatagattttatcttctacttttttcttttctggggctcttagcccttggtgtttcattgttgtgcaatgcaatcacaggaattacacttttaagagttaaatttaaaagtcagcagcacagacaaggcagatctcatcatttggaaatggtaatccagctcctggcgataatgtgtgtctcctgtatttgttggagcccatttctgggatacagaataattttgaatgggaaagagaaatataaagtatatgaagagcaaagtgatttcttacataggttacaatggccaacattggaa

SEQ ID NO: 4 - protein, 272 aa, Homo sapiens
NRLSVFFSVIFMTVGILSNSLAIAILMKAYQRFRQKSKASFLLLASGLVITDFFGHLINGAIAVFVYASDKEWIRFDQSNVLCSIFGICMVFSGLCPLLLGSVMAIERCIGVTKPIFHSTKITSKHVKMMLSGVCLFAVFIALLPILGHRDYKIQASRTWCFYNTEDIKDWEDRFYLLLFSFLGLLALGVSLLCNAITGITLLRVKFKSQQHRQGRSHHLEMVIQLLAIMCVSCICWSPFLGYRIILNGKEKYKVYEEQSDFLHRLQWPTLE

SEQ ID NO: 5 - DNA, 1005 bp, Pan troglodytes
aaccggctttccgtatttttttcagtaatcttcatgacagtgggaatcttgtcaaacagccttgccatcgccattctcatgaaggcatatcagagatttagacagaagtccaaggcatcgtttctgcttttggccagtggcctggtaatcactgatttctttggccatctcatcaatggagccatagcagtatttgtatatgcttctgataaagaatggatccgctttgaccaatcaaatgtcctttgcagtatttttggtatctgcatggtgttttctggtctgtgcccacttcttctaggcagtgtgatggccattgagcggtgtattggagtcacaaaaccaatatttcattctacgaaaattacatccaaacatgtgaaaatgatgttaagtggtgtgtgcttgtttgctgttttcatagctttgctgcccatccttggacatcgagactataaaattcaggcgtcgaggacctggtgtttctacaacacagaagacatcaaagactgggaagatagattttatcttctacttttttcttttctggggctcttagcccttggtgtttcattgttgtgcaatgcaatcacaggaattacacttttaagagttaaatttaaaagtcagcagcacagacaaggcagatctcatcatttggaaatggtaatccagctcctggcgataatgtgtgtctcctgtatttgttggagcccatttctggttacaatggccaacattggaataaatggaaatcattctctggaaacctgtgaaacaacactttttgctctccgaatggcaacatggaatcaaatcttagatccttgggtatatattcttctacggaaggctgtccttaagaatctctataagcttgccagtcgatgctgtggagtgcatgtcatcagcttacatatttgggagcttagttccattaaaaattccttaaaggttgctgctatttctgagtcaccagttgcagagaaatcagcaagcacctag

SEQ ID NO: 6 - protein, 334 aa, Pan troglodytes
NRLSVFFSVIFMTVGILSNSLAIAILMKAYQRFRQKSKASFLLLASGLVITDFFGHLINGAIAVFVYASDKEWIRFDQSNVLCSIFGICMVFSGLCPLLLGSVMAIERCIGVTKPIFHSTKITSKHVKMMLSGVCLFAVFIALLPILGHRDYKIQASRTWCFYNTEDIKDWEDRFYLLLFSFLGLLALGVSLLCNAITGITLLRVKFKSQQHRQGRSHHLEMVIQLLAIMCVSCICWSPFLVTMANIGINGNHSLETCETTLFALRMATWNQILDPWVYILLRKAVLKNLYKLASRCCGVHVISLHIWELSSIKNSLKVAAISESPVAEKSAST

SEQ ID NO: 7 - DNA, 1026 bp, Canis familiaris
aaccggctttcggtatttttttcagtaatcttcatgacagtgggaatcttgtcaaacagccttgccattgctattctcatgaaggcttatcagagatttagacagaagtccaaggcatcgtttctgcttttggccagtgccctggtaatcacagacttctttggccacctcatcaatggagctatagcagtctttgtgtacgcttccgataaagactggatccgctttgaccagtcgaacatcctttgcagtatctttggtatctgcatggtgttctccggtctgtgcccactttttctaggcagtgtgatggccattgagcgctgtatcggagtcaccaaaccaatatttcattctacgaaaattacatccaaacatgtgaaaatgatgttgagtggagtgtgcttgtttgccattttcatagctttgctacccatccttgggcatcgaaactataaaattcaagcatcgaggacctggtgtttctacaaaacagaacacatcaaagactgggaagataggttttaccttctgctcttttcttttctggggctcttagcccttggcgtttcattcttgtgcaatgccatcacaggaatcacacttttaagagtcaaatttaaaagtcagcagcacagacaaggcaggtctcatcattttgaaatggtcatccagctcctggctataatgtgtgtctcctgcatttgctggagtccatttctggtgacaatggccaacattggaataaatggagatcattctctggagacctgtaaaacaatactttttgctctccgaatggcaacgtggaatcaaatcttagatccttgggtgtacattctgctgcggaaggccgtccttaagaatctctacaaacttgccagacgctgctgtggagtgcatgtcatcagcttgcatgggtgggagcttagctccattaaaaattctctaaaggttgctgctatttctgagtctccagttacagagaaaataaatcagcaagcacctggtttaataggacagtaa

SEQ ID NO: 8 - protein, 341 aa, Canis familiaris
NRLSVFFSVIFMTVGILSNSLAIAILMKAYQRFRQKSKASFLLLASALVITDFFGHLINGAIAVFVYASDKDWIRFDQSNILCSIFGICMVFSGLCPLFLGSVMAIERCIGVTKPIFHSTKITSKHVKMMLSGVCLFAIFIALLPILGHRNYKIQASRTWCFYKTEHIKDWEDRFYLLLFSFLGLLALGVSFLCNAITGITLLRVKFKSQQHRQGRSHHFEMVIQLLAIMCVSCICWSPFLVTMANIGINGDHSLETCKTILFALRMATWNQILDPWVYILLRKAVLKNLYKLARRCCGVHVISLHGWELSSIKNSLKVAAISESPVTEKINQQAPGLIGQ

SEQ ID NO: 9 - DNA, 1026 bp, Mus musculus
aaccggctttcagtatttttttctatcatcttcatgacagtggggatcttatcgaacagcctggccatcgccattctcatgaaggcctaccagagatttagacagaagtcaaaggcttccttcctgcttttggctagtggcctggtgatcacagacttctttggccaccttatcaacggaggcatagctgtctttgtatatgcttctgataaagactggatccgctttgatcagtcaaacatcctgtgcagtatttttggaatctccatggtcttttctggcttgtgcccactttttctaggcagtgcgatggccatcgagaggtgtataggagtcaccaatcctatatttcactctacgaagatcacatctaaacacgtgaaaatgatcctgagtggtgtgtgcatgtttgctgtgttcgtggctgtgctgcccatccttggacaccgagattatcaaatccaagcgtccagaacctggtgtttctacaacacagagcacatcgaagactgggaagacagattttatctcctgttcttttctttcctcggactcttagctcttggtgtttccttctcgtgcaatgccgtcacgggagtcacactcttaagagtgaagttcagaagccagcagcataggcaaggcagatctcaccacctggagatgatcattcagctcctggccataatgtgcgtctcctgcgtctgctggagtccctttctggtaacaatggccaacattgcaataaatggaaataattccccagtgacctgtgaaacgacactttttgctctccgcatggcaacgtggaatcagatcttagatccctgggtctatattctgctacggaaggctgtccttaggaacctgtataaacttgccagtcgttgctgtggagttaacatcatcagcttgcatatctgggaactcagctccatcaagaattccttaaaggttgctgctatttctgagtcaccagctgcagagaaggagagccagcaagcatcgagtgaagctggactgtaa

SEQ ID NO: 10 - protein, 341 aa, Mus musculus
NRLSVFFSIIFMTVGILSNSLAIAILMKAYQRFRQKSKASFLLLASGLVITDFFGHLINGGIAVFVYASDKDWIRFDQSNILCSIFGISMVFSGLCPLFLGSAMAIERCIGVTNPIFHSTKITSKHVKMILSGVCMFAVFVAVLPILGHRDYQIQASRTWCFYNTEHIEDWEDRFYLLFFSFLGLLALGVSFSCNAVTGVTLLRVKFRSQQHRQGRSHHLEMIIQLLAIMCVSCVCWSPFLVTMANIAINGNNSPVTCETTLFALRMATWNQILDPWVYILLRKAVLRNLYKLASRCCGVNIISLHIWELSSIKNSLKVAAISESPAAEKESQQASSEAGL

SEQ ID NO: 11 - DNA, 26 bp, Artificial Sequence
  chemically synthesized primer
tcaccaatcctatatttcactctacg

SEQ ID NO: 12 - DNA, 21 bp, Artificial Sequence
  chemically synthesized primer
gagctgagttcccagatatgc

(19) United States
(12) Patent Application Publication
(10) Pub. No.: US 2011/0172308 A1
(43) Pub. Date: Jul. 14, 2011

(54) Antagonsim of PGF2a Receptor to Treat Hyertension Characterized by Activation of the Renin-Angiotensin-Aldosterone System

(75) Inventors: Garret A. Fitzgerald, Wayne, PA (US); Ying Yu, Shanghai (CN); Margaret Lucitt, Co-Kerry (IE)

(73) Assignee: The Trustees of The University of Pennsylvania, Philadelphia, PA (US)

(21) Appl. No.: 12/920,789

(22) PCT Filed: Mar. 9, 2009
(86) PCT No.: PCT/US09/36546

§ 371 Date: Mar. 28, 2011

Related U.S. Application Data
(60) Provisional application No. 61/068,502, filed on Mar. 7, 2008.

(51) Int. Cl. A61K 31/5575; A61P 9/12; A61P 35/00; C12Q 1/02

(52) U.S. Cl. 514/563; 514/561; 514/573; 435/29

(57) ABSTRACT

The present invention relates to methods for identifying candidate therapeutics for hypertension. The invention further provides a method for treating hypertension and/or decreasing atherogenesis by administration of an inhibitor of PGF_{2α} receptor.

### BACKGROUND OF THE INVENTION

Blood pressure (BP) regulation involves three components: 1) the nervous system; 2) the renin-angiotensin-aldosterone (RAA) system; and 3) kidney-fluid system. The nervous system is the first line control of blood pressure. The RAA system is the intermediate pressure controller. The kidney-fluid system mediates the long-term pressure control.

The RAA system plays an important role in the maintenance of vascular tone, circulating blood volume, and electrolyte balance in the body. Renin is a rate-limiting enzyme involved in the activation of the RAA system. Renin is secreted from the granular cells of juxtaglomerular apparatus (JGA) in the kidney. It converts plasma angiotensinogen to Ang I, which is successively changed to Ang II, a powerful vasoconstrictor, by angiotensin-converting enzyme (ACE) present on the epithelial cells of pulmonary vasculatures. Ang II acts on the adrenal cortex and stimulates the secretion of aldosterone, which facilitates sodium reabsorption in the kidney and expands the circulating blood volume. Thus, renin, Ang II and aldosterone are thought to be important players in the control of BP.

Renin secretion is precisely regulated through two major sensing mechanisms, along with regulation by the sympathetic nervous system. One mechanism is the baroreceptor mechanism, which senses the reduction in renal perfusion pressure and increases renin secretion (Hackenthal et al., 1990, Physiol Rev. 70:1067-1116). The other is the macula densa mechanism, which senses the decrease in the concentration of chloride ions in glomerular filtrate at the macula densa cells and increases renin secretion (Hackenthal et al, 1990, ibid). The macula densa cell, a differentiated tubular epithelial cell, is one of those composing the JGA. These two sensing mechanisms transmit their information to the granular cells via the respective mediators.

Members of the family of prostanoids, made up of prostaglandins and thromboxanes, are generated via COX-mediated metabolism of arachidonic acid. These lipid mediators exhibit wide-ranging biological actions that include regulating both vasomotor tone and renal sodium excretion. Prostacyclin (PGI₂) and PGE₂, but not PGF_{2α}, have been shown to be potent renin secretagogues (Jensen et al., 1996, Am. J. Physiol. 271:F659-F669; Fujino et al., 2004, J Clin Invest. 114(6): 805-812).

It is well known that excessive activation of the renin-angiotensin-aldosterone (RAA) system can lead to hypertension. Hypertension, commonly referred to as high blood pressure, is a medical condition in which the blood pressure of an individual is chronically elevated. Persistent hypertension is one of the risk factors for strokes, heart attacks, heart failure and arterial aneurysm, and is a leading cause of chronic renal failure. Even moderate elevation of arterial blood pressure leads to shortened life expectancy.

The prevalence of hypertension is significant. In the US population, approximately 1 in 5 adults is believed to have hypertension. In 2002, worldwide prevalence was estimated at over 600 million people. Current hypertension treatments, including ACE inhibitors, angiotensin II receptor antagonists, alpha blockers, beta blockers, calcium channel blockers, direct renin inhibitors and diuretics, target different points of the RAA system. Resistant hypertension, however, has become an increasing problem. Thus, new treatments for hypertension are of great medical value.

There exists a need in the art for new targets of therapy for hypertension and for new treatments for hypertension. The present invention addresses these needs.

### BRIEF SUMMARY OF THE INVENTION

The invention provides in part a method of treating hypertension in an individual. The method comprises administering a therapeutically effective amount of an inhibitor of prostaglandin F_{2α} receptor to an individual in need thereof, wherein the inhibitor decreases blood pressure in the individual. In one embodiment, the hypertension is renin-dependent hypertension.

The invention also provides a method of alleviating atherogenesis in an individual. The method comprises administering a therapeutically effective amount of an inhibitor of prostaglandin F_{2α} receptor to the individual in need thereof, wherein the inhibitor decreases blood pressure, thereby alleviating atherogenesis in the individual.

In some embodiments of the therapeutic methods of the invention, the inhibitor of prostaglandin F_{2α} receptor is a selective inhibitor. In some embodiments, the inhibitor is selected from AL-8810 (11 beta-fluoro-15-epi-15-indanyl-tetranor PGF_{2α}), PGF_{2α} dimethylamide, PGF_{2α} dimethylamine, and derivatives thereof.

The invention further provides a method of identifying a candidate therapeutic for the treatment of hypertension. The method comprises identifying an inhibitor of prostaglandin F_{2α} receptor, thereby identifying a candidate therapeutic for the treatment of hypertension. Optionally, the hypertension is renin-dependent hypertension. In one embodiment, the step of identifying an inhibitor of prostaglandin F_{2α} receptor comprises measuring a first level of prostaglandin F_{2α} receptor activity present in a cell in the presence of a prostaglandin F_{2α} receptor agonist; administering a test compound to the cell; and measuring a second level of prostaglandin F_{2α} receptor activity in the cell. A test compound that reduces the second level of prostaglandin F_{2α} receptor activity compared to the first level of prostaglandin F_{2α} receptor activity is identified as a candidate therapeutic for the treatment of hypertension. In one embodiment, the cell is a juxtaglomerular apparatus cell. In one embodiment, the juxtaglomerular apparatus cell is human. In another embodiment, the cell is a non-human mammal juxtaglomerular apparatus cell that recombinantly expresses human prostaglandin F_{2α} receptor. In some embodiments, the prostaglandin F_{2α} receptor activity is selected from the group consisting of: renin expression, renin release, plasma renin activity (PRA), plasma Ang II, plasma aldosterone concentration (PAC) and intracellular Ca²⁺.

Also provided is another method of identifying a candidate therapeutic for the treatment of hypertension. The method comprises measuring a first level of prostaglandin F_{2α} receptor activity present in a non-human mammal that expresses prostaglandin F_{2α} receptor in juxtaglomerular cells in the presence of a prostaglandin F_{2α} receptor agonist; administering a test compound to the non-human mammal; and measuring a second level of prostaglandin F_{2α} receptor activity in the non-human mammal. A test compound that reduces the second level of prostaglandin F_{2α} receptor activity compared to the first level of prostaglandin F_{2α} receptor activity is identified as a candidate therapeutic for the treatment of hypertension. In one embodiment, the hypertension may be renin-dependent hypertension. In some embodiments, the prostaglandin F_{2α} receptor activity is selected from the group consisting of: renin expression, renin release, plasma renin activity (PRA), plasma Ang II, plasma aldosterone concentration (PAC), blood pressure, AT1 expression, arginine vasopressin expression, and intracellular Ca²⁺. In some embodiments, the non-human mammal recombinantly expresses human prostaglandin F_{2α} receptor. In some embodiments, the non-human mammal is transgenic for human prostaglandin F_{2α} receptor. Optionally, the recombinant human prostaglandin F_{2α} receptor is expressed in juxtaglomerular apparatus cells.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIGS. 1A-1E depict blood pressure data. FIG. 1A depicts data for normalipodemic mice. WT=wild type. FP^{-/-}=prostaglandin F_{2α} receptor knockout. FIG. 1B depicts data for hyperlipidemic mice. Ldlr^{-/-}=Ldl receptor (Ldlr) knockout. Ldlr^{-/-}/FP^{-/-}=double knockout. FIG. 1C depicts systolic blood pressure (SBP) measured by tail cuff in FP^{-/-} mice on a normal chow diet and after salt depletion. FIG. 1D depicts SBP measured by tail cuff in Ldlr^{-/-}FP^{-/-} on a high fat diet after 12 weeks. FIG. 1E depicts SBP measured by telemetry in Ldlr^{-/-}FP^{-/-} on a high fat diet after 24 weeks. Values represent mean±SEMs analyzed by unpaired Student's t test.

FIGS. 2A-2E depict data related to renin-angiontensin-aldosterone system activation in WT and FP knockout mice under normal and salt depleted conditions. Values represent mean±SEMs analyzed by unpaired Student's t test. FIGS. 2A, 2B and 2C respectively depict data for PRA=plasma renin activity; Plasma Ang I=plasma angiotensin I; PAC=plasma aldosterone concentration. FIGS. 2D and 2E depict PRC and PAC following 12 and 24 weekd of high fat diet feeding in Ldlr^{-/-} and Ldlr^{-/-}/FP^{-/-} mice. For FIGS. 2D and 2E, *P<0.05, **P<0.01 versus Ldlr^{-/-} control (n=10-14).

FIGS. 3A-3C depicts data related to angiotensin II receptor (AT1) expression and blood pressure effects of Ang II injection in WT and FP knockout mice. FIG. 3A depicts AT1a and FIG. 3B depicts AT1b gene expression in aorta by real time RT-PCR. *p<0.01 (n=4) versus WT, repeated 3 times. FIG. 3C depicts blood pressure (BP) after Angiotengin II (Ang II) administration (50 ng/kg) compared with baseline in FP^{-/-} and WT littermates. Mean arterial BP was monitored in mice by pressure transducer connected to a catheter in left artery. Baseline BP is readout before injection. BP changes are depicted as difference between baseline and after injection at one min intervals. *p<0.01 versus WT, n=8-9.

FIGS. 4A-4E depict expression data for FP in different sections of kidney. FIG. 4A is a schematic drawing of a kidney. FIG. 4B depicts FP mRNA expression detected by RT-PCR in microdissected mouse nephron segments. RT + and − are presence and absence ore reverse transcriptase;—is control to verify no PCR contamination. Cortical Collecting Duct (CCD); Proximal Convoluted Tubule (PCT); Preglomerular Arteriole (PGA); glomerulus (Glom); and positive control (kidney cortex). FIG. 4C is a graph of the data shown in FIG. 4B. FIG. 4D depicts quantification of juxtagomerular granular (JG) negative and positive afferent arterioles in WT and FP^{-/-} mice. FIG. 4E depicts renin gene expression in kidney from FP^{-/-} and WT mice. RNA extracted from whole kidney was subjected to realtime RT-PCR. Data represents mean±SEMs, *p<0.01 (n=4), repeated 3 times.

FIGS. 5A-5E depict data related to renin expression resulting from activation of FP or prostacyclin (IP) receptor in in vitro cultures of juxtaglomerular apparatus cells obtained from WT or FP knockout mice. FIG. 5A is a graph depicting the effect of FP agonists Latanoprost (Lata) and Travoprost (Trav), and IP agonist Cicaprost (Cica) on renin mRNA expression in JG enriched cultured cells. Each point represents mean±SEMs of 4 cell groups, repeated 3 times. FIG. 5B is a bar graph illustrating the effect of effect of Latanoprost and Cicaprost on cAMP content in cultured cells rich in JG cells. Data are presented as cAMP fold change from 3-6 independent samples (6-well plate) from 3 independent experiments. *, p<0.05 vs vehicle controls, n=3. FIG. 5C is a graph of the original recording of membrance capacitance in a single mouse JG cell from WT mice. FIG. 5D is a bar graph exhibiting the relative change of membrane capacitance for JG cells stimulated with 1 μM Latanoprost. FIG. 5E is a graph of whole cell currents were measured in response to 11 pulses from −150 to +90 mV in 30-mV steps for 200 ms from a holding potential of −30 mV. Pulses were applied before and after superfusion with 1 μM Latanoprost. Mean steady-state current-voltage curves from 4 independent experiments before (cycle) and after (square) 20 min of capacitance measurements are given.

FIGS. 6A-6I depict data related to FP expression and systemic blood pressure in WT and FP knockout mice. FIG. 6A is an image depicting FP expression detected in the medial layer of renal resistant artery in FP^{+/-} by β-gal activity (arrows point to representative staining). FIGS. 6B and 6C are images depicting FP expression (via X-gal staining) in normal aorta from FP^{-/-} mice (6B) and lesion aorta from Ldlr^{-/-}/FP^{-/-} mice, with nuclear red staining (arrows indicate representative nuclear red staining). Rectangle boxed area is shown on the right at higher magnification. FIGS. 6D-6G are images of aorta (6D, 6E) and aortic root (6F, 6G) sections from Ldlr^{-/-} mice on normal chow (6D, 6F) or high fat diet (HFD; 6E, 6G) for a period of 24 weeks with Hoechst nuclear staining (light gray) subjected to in situ hybridization of positive antisense probe (top panels) and negative control sense probe (bottom panels). Scale bars 100 um. FIGS. 6H and 6I are graphs depicting the effect of effect of PGF_{2α} infusion on systemic blood pressure in WT and FP knockout mice. FIG. 6H is a representative blood pressure (BP) tracing from FP^{-/-} and WT littermates at different doses of PGF_{2α} infusion, marked by arrows. FIG. 6I is a graph of mean arterial BP increase after PGF_{2α} administration in FP^{-/-} and WT mice. The BP increase is the difference between peak/trough after injection and baseline. Value are presented as mean±SEMs.

FIGS. 7A-7G depict data related to water metabolism in WT and FP knockout mice. Values represent mean±SEMs analyzed by unpaired Students t-test. FIG. 7A is a bar graph of baseline water consumption of WT and FP −/− mice. Male and female mice (8-12 wks old) housed in metabolic cages for 4 days prior to measurements; mice were allowed free access to food and water throughout the study. *P<0.05 versus WT, n=10-11. FIG. 7B is a bar graph of FP receptor expression in the hypothalamus detected by qRT-PCR. FIG. 7C is a bar graph of hypothalamic arginine-vasopressin (AVP) mRNA expression is upregulated in FP −/− mice. *P<0.05 versus WT controls (n=4), repeated 3 times. FIG. 7D is a bar graph of urine osmolality in FP^{-/-} mice. *P<0.05 versus WT, n=8-10. FIG. 7E is a bar graph of plasma osmolality in FP^{-/-} and WT mice. Water deprivation (WD) initiated by removal of water bottles from cages, the plasma osmolality was measured in mice before and following 24 hrs of WD. *P<0.05 versus WT, n=6-7. FIG. 7F is a bar graph of medullary osmolality in FP^{-/-} and WT mice. FIG. 7G is a bar graph of urine output in FP^{-/-} mice.

FIGS. 8A-8C depict data related to extent of atherogenesis in from male and female Ldlr knockout and Ldlr/FP double knockout mice. Values are presented as mean±SEM and analyzed by unpaired Student's t test. FIGS. 8A and 8B are bar graphs of atherosclerotic lesion area in female (8A) and male (8B) Ldlr mice and Ldlr^{-/-}/FP^{-/-} mice at 12 and 24 weeks on a high fat diet. Atherosclerotic lesion area measured by en face staining. **P<0.01 (n=12-16) versus Ldlr^{-/-} control mice. FIG. 8C is a bar graph of lesion area in aortic root sections from male mice on a high fat diet for 24 weeks stained using H&E and lesional area calculated. *P<0.05 (n=8-10) versus Ldlr^{-/-} control mice.

FIG. 9 comprises images of tissue sections obtained from Ldlr^{-/-} (positive staining) or Ldlr^{-/-}/FP^{-/-} mice (aortic arch and root), stained for expression of LacZ. Arrows indicates regions of positive staining for expression of LacZ in the positive control. Boxed regions in images on left are magnified in images on the right.

FIGS. 10A-10D are related to cellular composition in FP deficient mice. FIG. 10A comprises images of representative aortic root sections (8 μm) from Ldlr^{-/-} and Ldlr^{-/-}/FP^{-/-} immunostained for surface macrophages using rat anti-mouse CD68. Scale bar 100 μm. FIG. 10B is a bar graph of the quantification of CD68 positive staining (brown) to total lesion area in Ldlr^{-/-} control and Ldlr^{-/-}/FP^{-/-} male mice on a high fat diet 24 weeks. ***P<0.0001 versus Ldlr^{-/-} control mice for immunostaining (n=8-10). FIGS. 10C and 10D are bar graphs of the quantitative analysis of aortic root sections (8 μm) from male Ldlr^{-/-} and Ldlr^{-/-}/FP^{-/-} mice on a high fat diet for 24 weeks, immunostained anti alpha-SMC actin brown, counterstained using hematoxylin (10C) or Massons Trichrome for collagen composition blue (10D). Values represent mean±SEMs (n=6-8).

FIGS. 11A-11C depict data related to expression of inflammatory cytokines in whole aortas from Ldlr^{-/-} control and Ldlr^{-/-}/FP^{-/-} mice on a high fat diet for 12 and 24 weeks. Expression was assessed by real time RT-PCR expression analysis. FIGS. 11A-11C depict bar graphs of data for TNF_{α}, TGF_{β} and inducible nitric oxide enzyme (iNOS), respectively, normalized to 18S rRNA. Values are presented as mean±SEM and analyzed by unpaired Student's t test. *P<0.05, **P<0.01 and ***P<0.0001 versus Ldlr^{-/-} control mice for qRT-PCR expression (n=6).

FIGS. 12A-12E 11C depict data related to expression of cytokines in peritoneal macrophage from Ldlr^{-/-} and Ldlr^{-/-}/FP^{-/-} mice. Peritoneal macrophages cultured in the presence (+) and absence (−) of 10 μg/ml LPS for 6 hrs. qRT-PCR expression analysis normalized to 18S rRNA. Cytokines assessed were: TNFα (12A); TGFβ (12B); IL 6 (12C); IL 12 (12D) and iNOS (12E). Values represent mean±SEMs (n=4).

### DETAILED DESCRIPTION OF THE INVENTION

The invention arises in part from the observation disclosed herein that PGF_{2α} and the PGF_{2α} receptor play a significant role in maintaining blood pressure. Specifically, PGF_{2α} elevates blood pressure, accelerates atherosclerosis and restrains water intake. In brief, mice that are knockouts (KO) for the PGF_{2α} receptor (FP) have lower blood pressure. Furthermore, there is decreased activation in the renin-angiotensin-aldosterone (RAA) system which may mechanistically be related to the reduction in blood pressure in FP KO mice. Additionally, FP KO retards atherogenesis in hyperlipidemic mice.

Consequently, the present application features a method for identifying candidate therapeutics for the treatment of hypertension or parthenogenesis. The invention further provides a method of treating hypertension, pre-hypertension or atherogenesis. The method comprises administering an inhibitor of PGF_{2α} receptor to an individual in need thereof.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

Standard techniques are used for the synthesis and manipulation of nucleic acid and peptides. The techniques and procedures are generally performed according to conventional methods in the art and various general references (e.g., Sambrook and Russell, 2001, Molecular Cloning, A Laboratory Approach, Cold Spring Harbor Laboratory Press, Cold Spring Harbor, N.Y.; Ausubel et al., 2002, Current Protocols in Molecular Biology, John Wiley & Sons, New York, N.Y.; Gerhardt et al. eds., 1994, Methods for General and Molecular Bacteriology, American Society for Microbiology, Washington, D.C.), which are provided throughout this document.

The articles “a” and “an” are used herein to refer to one or to more than one (i.e., to at least one) of the grammatical object of the article. By way of example, “an element” means one element or more than one element.

As used herein, “hypertension” refers to chronically elevated blood pressure. Current medical guidelines define hypertension as a systolic blood pressure >140 mm Hg, a diastolic blood pressure >90 mm Hg, or both. However, should the medical guidelines change, the invention is intended to encompass hypertension as so revised.

As used herein, “pre-hypertension” refers to chronic blood pressure between 120/80 mmHg to 139/89 mmHg. Per-hypertension is a clinical designation chosen to identify individuals at high risk of developing hypertension.

As used herein, the term “renin-dependent hypertension” means hypertension characterized by the abnormal activation of the renin-angiotensin-aldosterone system.

As used herein, a disease or disorder is “alleviated” or “treated” if the severity of a symptom of the disease or disorder, the frequency with which such a symptom is experienced by a patient, or both, are reduced. For instance, as used herein, “treating hypertension” and “treating pre-hypertension” means reducing the chronic blood pressure experienced by a patient. “Alleviating atherogenesis” refers herein to reducing the rate and/or extent of atherogenesis.

PGF_{2α} is used herein to refer to prostaglandin F_{2α}.

As used herein, “PGF_{2α} receptor” refers to the G-protein coupled rhodopsin-type receptor that specifically binds PGF_{2α}. PGF_{2α} receptor is herein abbreviated as FP.

As used herein, “PGF_{2α} receptor inhibitor compound” or “PGF_{2α} receptor inhibitor” refers to a compound which inhibits PGF_{2α} receptor activity induced by the binding of an agonist of the PGF_{2α} receptor, such as the naturally-occurring ligand, PGF_{2α}. Such an inhibitor may be a competive inhibitor (competes for binding to the PGF_{2α} binding site) or a non-competitive inhibitor. A non-competitive inhibitor binds to a site other than the PGF_{2α} binding site and inhibits thereby PGF_{2α} receptor activity. A non-competitive inhibitor may bind directly to a PGF_{2α} receptor or may bind to a molecule functionally associated with the PGF_{2α} receptor. “Functionally associated” refers to molecules that play a role in the activity induced by a naturally-occurring ligand of the PGF_{2α} receptor. Preferably, a functionally associated molecule also physically contacts the PGF_{2α} receptor.

As used herein, “therapeutically effective amount” refers to a nontoxic but sufficient amount of an agent to provide the desired biological result. That result can be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in an inducible manner.

A “constitutive promoter” is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

“Cardiovascular risk” is used herein to refer to the likelihood or possibility of incurring or experiencing a cardiovascular event. A decrease in cardiovascular risk is a relative term and refers to a reduced likelihood or possibility of a cardiovascular event compared to the risk under different conditions or to a normal population risk. With regard to cardiovascular risk and a medicament, the risk can be assessed relative to a patient's own risk when not taking the medicament, or with respect to a population that does not have clinical evidence of a cardiovascular disease and/or is not at risk for a cardiovascular event and is not taking the medicament. The population may be representative of the patient with regard to approximate age, age group and/or gender.

“Cardiovascular event” as used herein refers to a disorder or disease of the cardiovascular system having a sudden onset; it can also refer to a sudden worsening of such a disorder or disease. Examples of cardiovascular events include, without limitation: cardiac arrest, myocardial infarction, thrombosis, deep vein thrombosis, pulmonary thrombosis, atherogenesis, atherosclerosis, plaque fracture, ischemia, stroke, worsening of angina, and congestive heart failure.

“Clinical evidence of cardiovascular disease” is used herein to refer to medical evidence indicative of cardiovascular disease, as established by American College of Cardiology guidelines current at the time of filing of this application. Such clinical evidence includes, but is not limited to, abnormal results from: blood pressure, blood tests including a lipid profile, high density cholesterol, low density, cholesterol, triglycerides, cardiac biomarkers (enzymes, proteins, and hormones, such as troponin, myoglobin, b-type natriuretic peptide and creatine phosphokinase, that are associated with heart function, damage or failure), electrocardiograms (ECG or EKG), stress tests, chest x-ray, MUGA scan, computed tomography (CT), nuclear scanning (nuclear heart scan), echocardiogram (heart ultrasound), cardiac catheterization (coronary angiography), duplex/doppler ultrasound, magnetic resonance angiography (MRA) and magnetic resonance imaging (MRI). Documented incidents of myocardial infarctions, heart attack or plaque-associated thrombus are also clinical evidence of cardiovascular disease.

By the term “specifically binds,” refers to a ligand that binds to its cognate binding partner in a sample, but does not substantially recognize or bind other molecules in a sample.

### DESCRIPTION OF THE INVENTION

The invention provides a method of identifying candidate therapeutics for treatment of hypertension. Inhibition of PGF_{2α} receptor activity, as demonstrated herein, is expected to result in a decrease in blood pressure, and therefore, may be useful as a therapeutic strategy in the reduction of chronic elevated blood pressure (hypertension). As shown here, inhibition of PGF_{2α} receptor activity has effects on the RAA system which may mechanistically relate to the reduction in blood pressure. As shown herein, inhibition of PGF_{2α} receptor activity reduces plasma renin concentration, plasma angiotensin concentration and plasma aldosterone concentration. Furthermore, atherogenesis is retarded by inhibition of PGF_{2α} receptor activity. Accordingly, methods of treating hypertension and of decreasing the rate and/or extent of atherogenesis are also provided.

Identifying Inhibitors of PGF_{2α} Receptor

Inhibitors of PGF_{2α} receptor activity may be identified by screening test compounds. For instance, inhibitors of PGF_{2α} receptor activity may be identified by screening test compounds in vitro using cells that express PGF_{2α} receptor. Nonlimiting examples of such cells include juxtaglomerular apparatus cells, cortical collecting duct cells, proximal convoluted tubule and luteal cells.

Generally, inhibitors are identified by assaying PGF_{2α} receptor activity in the presence of a PGF_{2α} receptor agonist of a cell that expresses PGF_{2α} receptor and a test compound, and comparing that level of activity to the level of activity in the absence of the test compound. A test compound that reduces PGF_{2α} receptor activity in the presence of an agonist is identified as an PGF_{2α} receptor inhibitor. The skilled artisan is knowledgeable about the appropriate control experiments necessary to determine if an inhibitor is selective and if it is a competitive inhibitor or non-competitive inhibitor.

PGF_{2α} receptor activity refers to any biological activity that results from activation of PGF_{2α} receptor by the binding of a PGF_{2α} receptor agonist, such as PGF_{2α} or fluprostenol. Non-limiting examples of such activity include, but are not limited to, changes in renin expression, renin release, and changes in intracellular Ca²⁺. Exemplary methods of assessing renin expression and renin release are described herein and are known in the art. Exemplary methods of assessing changes in intracellular Ca²⁺ are known in the art (see, for instance, Abramovitz et al., 1994, JBC 269:2632-2636 and Kelly et al., 2003, J Pharm Exper Therapeutics 304:238-245).

Expression of PGF_{2α} receptor may be from an endogenous PGF_{2α} receptor gene. Alternatively, the PGF_{2α} receptor may be expressed from a heterologous gene introduced into the cell by recombinant methods. The introduced heterologous nucleic acid may be present transiently, or may be present stably in the cell, for instance due to insertion into the cell's chromosomal material. Cells useful for recombinant expression of PGF_{2α} receptor include, but are not limited to, Xenopus oocytes. Expression of the endogenous or heterologous gene may be constitutive or inducible.

The skilled artisan is familiar with the many methods of introducing heterologous nucleic acid into a cell, as well as the sequence elements necessary for transcription and translation of a coding sequence. See, for instance, Sambrook et al. (2001, Molecular Cloning: A Laboratory Manual, Cold Spring Harbor Laboratory Press, Cold Spring Harbor, N.Y.), in Ausubel et al. (eds., 1997, Current Protocols in Molecular Biology, John Wiley & Sons, New York), and in Gerhardt et al. (eds., 1994, Methods for General and Molecular Bacteriology, American Society for Microbiology, Washington, D.C.). In a preferred embodiment of the method for identifying inhibitors of PGF_{2α} receptor, the PGF_{2α} receptor coding sequence, or variant thereof, is from the same organism that is the intended recipient of treatment with the so-identified PGF_{2α} receptor inhibitor.

PGF_{2α} receptor coding sequences have been obtained and sequenced in several organisms, and any one can be used in the instant invention. PGF_{2α} receptor coding sequences useful in the instant invention include, but are not limited to: human PGF_{2α} receptor isoform 1 (mRNA, NCBI GenBank® Accession number NM_{—}000959, SEQ ID NO. 1; protein, NCBI GenBank® Accession number NP_{—}000950, SEQ ID NO. 2), and isoform 2 (mRNA, NCBI GenBank® Accession number NM_{—}001039585, SEQ ID NO. 3; protein, NCBI GenBank® Accession number NP_{—}001034674, SEQ ID NO. 4), chimpanzee PGF_{2α} receptor (mRNA, NCBI GenBank® Accession number XM_{—}513513, SEQ ID NO. 5; protein, NCBI GenBank® Accession number NP_{—}513513, SEQ ID NO. 6), dog PGF_{2α} receptor mRNA, (NCBI GenBank® Accession number NM_{—}001048097, SEQ ID NO. 7; protein, NCBI GenBank® Accession number NP_{—}001041562, SEQ ID NO. 8), and mouse PGF_{2α} receptor (mRNA, NCBI GenBank® Accession number NM_{—}008966, SEQ ID NO. 9; protein, NCBI GenBank® Accession number NP_{—}032992, SEQ ID NO. 10). Furthermore, any sequence encoding a variant PGF_{2α} receptor protein can be used, provided the PGF_{2α} receptor variant protein retains the activity of naturally-occurring PGF_{2α} receptor. Methods for assessing PGF_{2α} receptor activity are discussed elsewhere herein.

The heterologous PGF_{2α} receptor coding sequence may be operably linked to other nucleic acid sequences. Nonlimiting examples of other nucleic acid sequences are inducible promoters and other coding sequences, such as protein tags. By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region. Inducible promoters are useful for controlled overexpression of the PGF_{2α} receptor coding sequence. The inducible promoter may be that normally linked to the PGF_{2α} receptor coding sequence or may be from another gene. Protein tags, such as affinity tags or epitopes, are useful, for instance, in simplifying purification of the fusion protein. Sequences of inducible promoters and protein tags are well known in the art to the skilled artisan.

Inhibitors of PGF_{2α} receptor activity can also be identified by screening test compounds using organisms, such as mice, that express PGF_{2α} receptor. The organism may express an endogenous PGF_{2α} receptor or a heterologous PGF_{2α} receptor. In one embodiment, expression of the endogenous PGF_{2α} receptor gene is reduced or eliminated by standard known to the skilled artisan, including but not limited to gene knockout, gene knock down and RNAi, and a heterologous PGF_{2α} receptor gene is introduced into a cell in the organism. The organism may be a transgenic animal. Preferably, the heterologous PGF_{2α} receptor gene encodes human PGF_{2α} receptor.

PGF_{2α} receptor activity may be assessed directly or indirectly in biological samples from the organism after exposure to a test compound in the presence of a PGF_{2α} receptor agonist, and compared to PGF_{2α} receptor activity in the absence of the test compound. A test compound that reduces PGF_{2α} receptor activity in the presence of a PGF_{2α} receptor agonist is identified as a PGF_{2α} receptor activity inhibitor. PGF_{2α} receptor activity may be assessed by renin expression, renin release, plasma renin activity, plasma Ang II, plasma aldosterone concentration, blood pressure, AT1 expression, arginine vasopressin expression, and intracellular Ca²⁺. The skilled artisan is knowledgeable about the appropriate control experiments necessary to identify that an inhibitor is a competitive or noncompetitive inhibitor of PGF_{2α} receptor. Suitable biological samples for detection of PGF_{2α} receptor activity directly or indirectly include, but are not limited to, kidney tissues, particularly JGA cells, blood (whole blood or plasma) and urine.

Advantageously, assaying test compounds for their capacity to inhibit PGF_{2α} receptor activity an organism enables biomarkers of cardiovascular risk to be measured as well. In particular, atherogenesis can be assessed in an organism. Decreasing hypertension by inhibiting PGF_{2α} receptor activity is believed to lead to a decrease in cardiovascular risk, in particular, a decrease in the rate or extent of atherogenesis.

Test compounds for use in the screening methods can be small molecules, nucleic acids, peptides, peptidomimetics and other drugs. The test compounds of the present invention can be obtained using any of the numerous approaches in combinatorial library methods known in the art, including biological libraries, spatially-addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam, 1997, Anticancer Drug Des. 12:145). Inhibitors of PGF_{2α} receptor activity identified by the inventive method may be useful directly in therapeutic applications, and may also serve as lead drugs in the development of further therapeutics.

Methods for Treatment

The present application features methods for alleviating hypertension, alleviating pre-hypertension, and alleviating atherogenesis. In some embodiments, the hypertension and pre-hypertension is renin-dependent. The method comprises administering a therapeutically effective amount of an inhibitor of PGF_{2α} receptor (FP) to an individual in need thereof. Preferably, the inhibitor selectively inhibits the PGF_{2α} receptor.

Individuals for whom the method is useful include any animals that are diagnosed with or suspected of hypertension, those at risk of developing (e.g., individuals diagnosed with pre-hypertension) and those diagnosed with or at risk of atherogenesis. Non-limiting examples of such animals are mammals, such as humans, non-human primates, cattle, horses, dogs, sheep, goats, mice, rats and pigs. Preferably, the individual is a human. Treatment according to the methods of the invention may be particularly useful for individuals with clinical evidence of atherogenesis or at risk for atherogenesis.

Hypertension or pre-hypertension may be diagnosed by methods well known in the art. Essential (or primary) hypertension refers to hypertension for which no specific medical cause can be found to explain a patient's condition. Secondary hypertension indicates that the high blood pressure is a result of (i.e., secondary to) another condition, such as kidney disease or tumors. More than 95% of individual diagnosed with hypertension have essential hypertension. A significant number of essential hypertension is renin-dependent hypertension. Hypertensive individuals whose plasma renin activity (PRA) is not inhibited are considered to have an inappropriate amount of renin and thus to have renin-dependent hypertension. Diagnosis of atherogenesis is accomplished using clinical methods known in the art.

PGF_{2α} receptor inhibitors include, but are not limited to, AL-8810 (11beta-fluoro-15-epi-15-indanyl-tetranor PGF_{2α}), PGF_{2α} dimethylamide and PGF_{2α} dimethylamine and functional derivatives thereof.

Preferably, the PGF_{2α} receptor is selective for inhibition of mPGES-1. Selective PGF_{2α} receptor inhibitors are those which inhibit PGF_{2α} receptor activity to a greater extent than other prostanoid receptors, such as, but not limited to, PGD₂ receptor, PGE₂ receptor and PGI₂ receptor. Preferably a PGF_{2α} receptor inhibitor has a selectivity ratio of PGF_{2α} receptor inhibition over non-PGF_{2α} receptor inhibition of at least about 5, more preferably about 10, and more preferably about 50.

Administration of PGF_{2α} Receptor Inhibitors

The therapeutic methods of the invention encompass the use of pharmaceutical compositions of an appropriate small molecule, protein or peptide and/or isolated nucleic acid that inhibits PGF_{2α} receptor to practice the methods of the invention. The pharmaceutical compositions useful for practicing the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal.

Typically dosages of a PGF_{2α} receptor inhibitor which may be administered to an animal, preferably a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

Controlled- or sustained-release formulations of a pharmaceutical composition of the invention may be made using conventional technology.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods used in the following experiments are now described.

Animal Husbandry: All animals were housed and procedures carried out according to Institutional Animal Care and Usage Committee (IACUC) of the University Of Pennsylvania guidelines. All the FP^{-/-} (Sugimoto et al., 1997, Science 277: 681-683) and littermate control mice for experiments are on a C57BL6 background. For atherosclerosis study, mice at 5-7 weeks age were placed on a high fat western style diet (Harlan Teklad 88137, 0.7% NaCl) for a period of 12 or 24 weeks.

Blood Pressure Measurement by Tail Cuff and Telemetry: Systolic blood pressure was measured in conscious mice (age and gender matched) by using both computerized noninvasive tail-cuff system (Visitech Systems) and PA-C20 telemetry probes (Data Sciences International) as described elsewhere (Guan et al. (2007, J Clin Invest. 117: 2496-2505).

En Face Quantification of Atherosclerosis Lesion Area: Formalin fixed aortas were cleaned of adventitial fat, opened longitudinally and stained with Sudan IV (Sigma Aldrich St. Louis, Mo.). The stained aorta was pinned onto black wax for quantification. Images were photographed and digitized using the Image Pro analysis system (Phase 3 Imaging systems). Cross-sectional analysis of lesion area burden was also carried out on H&E stained 8 μm aortic root sections using Phase 3 Imaging systems. Total lesion area over 300 μm of the aortic root was measured every 96 μm, average lesional area derived from 3-4 sections.

Culture of Juxtaglomerular (JG) cells: JG cells were prepared as described (Fujino et al., 2004, J Clin Invest. 114: 805-812). The kidneys were removed, decapsulated, minced with a surgical blade, incubated with 0.1% collagenase for 15 minutes at 37° C. and filtered through a 40 μm nylon mesh. The filtered cells were placed on 15 ml of 30% isosmotic Percoll solution (Amersham Biosciences) and centrifuged at 27,000 g for 20 minutes at 4° C. The cell layer near the surface, enriched with JG cells, was collected and washed. The cells were then plated onto a collagen-coated dish (60 mm) in 5 ml of culture medium: RPMI-1640 supplemented with 2% FBS, transferrin (10 μg/ml), insulin (10 μg/ml), sodium selenite (0.67 ng/ml), penicillin (50 U/ml), and streptomycin (50 μg/ml) (Invitrogen Corp.). The cells were cultured for 20 hours in a humidified atmosphere containing 5% CO2 at 37° C. After the culture medium was changed to a fresh one containing indomethacin (10 μM), various concentrations of Cicaprost (Schering AG), an IP agonist, or Latanoprost and Travoprost, FP agonists (Cayman Chemical Co.) were added, and the cells were incubated for a further 20 hours. After the cell culture, total RNA was prepared, and RT-PCR analyses for renin mRNA expression were performed.

Measurement of Plasma Renin Concentration and Plasma Aldosterone Concentration: Blood was collected for PRC by cardiac puncture, and EDTA (0.01M pH 8.0) was added. After centrifugation at 1,500 g for 10 minutes, plasma was collected and stored at −80° C. until use. To determine PRC, plasma (10 μl) was incubated for 1 hour at 37° C. with 10 μl of plasma prepared from nephrectomized mice 30 hours after the operation plus 10 μl of phosphate buffer (50 mM, pH 6.6). The generated Ang I was measured by an enzyme immunoassay (EIA) kit (Peninsula Laboratory Inc.). Residual renin activity in plasma from nephrectomized mice was subtracted from the PRA of each sample. The PAC was measured by an EIA kit (Cayman Chemical Co.).

Mean Arterial Blood Pressure (BP) Measurements: The right internal jugular vein and left carotid artery of anesthetized mice were cannulated with PE-10 tubing. The arterial catheter was connected to a Capto SP844 pressure transducer, and BP was monitored continuously with a PowerLab/8SP system. Mice were injected via the right internal jugular vein with Ang I (50 ng/kg) or PGF_{2α} (10, 30, 90, 270 μg/kg). The same volume of saline was injected before Ang I or PGF_{2α} administration to exclude volume-mediated BP changes.

Visualization of JG cell granules: Kidneys were treated with 5 M HCl for 1 hour at 37° C. After acid removal, kidneys were kept in distilled water for 48 hours at 4° C. Vascular trees were microdissected under a stereomicroscope, and the JG cells were directly visualized by phase contrast as previously described (Casellas et al., 1993, Am J Physiol. 265: F151-156). In 12 vessel trees from each genotype, the number of afferent arterioles were counted with and without JG granularity.

Plasma and Serum Collection: Mice were fasted 24 hours prior to blood collection and were anesthetized with CO₂. Samples approx 1 ml were collected by cardiac puncture at the time of sacrifice and split in two for serum and plasma isolation. Serum was isolated by placing in Sarstedt Microvette (20.1308.100) tubes containing clot activator and spun at 10,000 g for 5 minutes at room temperature in a table top micro centrifuge and stored at −80° C. Plasma was isolated by placing in Sarstedt Microvette (20.1309.100) tubes containing heparin and spun at 2,000 g for 5 minutes at room temperature in a table top micro centrifuge and stored at −80° C.

Cholesterol measurement: Total cholesterol levels in serum samples were measured enzymatically on a Cobas Fara II autoanalyzer (Roche Diagnostic Systems Inc, Nutley, N.J.) with reagents from Wako Chemicals (Richmond, Va.).

Salt depletion: Mice were fed with normal diet containing 0.70% NaCl or low-salt diet containing 0.12% NaCl (Harlan Teklad.) as previously reported (Fujino et al., 2004, J Clin Invest. 114: 805-812). Mice on a low salt diet also received an intraperitoneal injection of furosemide (25 mg/kg) for 7 days.

Water deprivation (WD): Adult mice (8-12 wk old) were transferred to metabolic cages and given free access to food and water for 3 days to acclimatize prior to determination of water intake and urine volume. Subsequently, urine volume and water consumption were monitored for 5 consecutive days. For WD experiments, mice were deprived of access to drinking water by removing the water bottles for 24 hours before euthanizing.

Medullary Osmolality determination: The kidneys were excised and the inner medullas were rapidly dissected, blotted onto Whatman 3M filter paper, and placed into preweighed microfuge tubes. Samples were weighed immediately and subsequently dried in an oven over a desiccant at 60° C. for 8 hours. After reweighing, 25 μl of deionized distilled water was added to each tube and immersed in a boiling water bath for 3 minutes. Tubes were then centrifuged briefly and incubated at 4° C. for 24 hours to allow for diffusion. After centrifugation for 1 minute at 8,000 g, the supernatant was analyzed for osmolality by freezing point depression and corrected for the original water content of the sample.

Macrophage preparation: Mice were injected intra-peritoneal with 0.5 ml of 10% thioglycollate media and sacrificed after 4 days. Peritoneal lavages were collected, filtered, centrifuged at 1,000 g at 4° C. for 10 minutes, and resuspended in phenol-free RPMI medium 1640 (Invitrogen Carlsbad, Calif.) supplemented with 10% FBS. Cells were plated onto six-well plates, incubated at 37° C. for 4 hours, and then changed into serum-free media overnight. Cells were treated the next day with fresh serum-free media with LPS (Sigma-Aldrich L-2654 from Escherichia coli 026:B6, 1 μg/ml in PBS) or PBS (control) and incubated for 6 hours. Cells were harvested for RNA expression analysis.

Histological Examination of Lesion Morphology: OCT compound-embedded 8 μm tissue sections were acetone fixed and peroxidase-quenched. Blocking step was carried out with 10% FBS followed by incubation with primary antibodies: 20 ug/mL (1:200) FITC-conjugated mouse anti-α-smooth muscle actin clone 1A4 (Sigma-Aldrich), 50 ug/mL (1:200) FITC-conjugated rat anti mouse macrophage CD68 (FA-11; Serotec) and 5 ug/ml (1:200) rat anti mouse CD31 (PECAM-1) (BD Pharmingen). Sections were then incubated with α Fluoro-POD, Fab fragment (Roche) for smooth muscle cell actin, biotinylated rabbit anti-rat (Vector BA-4001) for macrophage and PECAM-1. Vectastain ABC avidin-biotin amplification (Vector Laboratories, Burlingame, Calif.) was used for macrophage and PECAM-1, and all sections developed with diaminobenzidine (Dako, Carpinteria, Calif.). Counterstaining was carried out with Harris modified hematoxylin (Sigma HHS16), and isotype controls were run in parallel with negligible staining observed in all cases. Massons Trichrome (Sigma-Aldrich) was used to stain for collagen content. Cross sectional quantitative immunostaining analysis for smooth muscle cell, macrophage foam cells and collagen content was performed every 96 μm over 300 μm of the aortic root of 6-8 mice for each group and expressed as a percentage of total lesional area.

In Situ Hybridization and β-galactosidase staining: The in situ probe for FP was generated from mouse kidney cDNA using primers (forward primer TCACCAATCCTATATTTCACTCTACG (SEQ ID NO. 11) and reverse primer GAGCTGAGTTCCCAGATATGC (SEQ ID NO. 12)) spanning exon 2 and 3 amplified a sequence length of 600 bp which was cloned into a PCR4 vector (TOPO TA Cloning Invitrogen). A restriction digest was set up with Ssp1 and Bsr G1 restriction enzymes (New England Biolabs), antisense riboprobe was generated using T₃ polymerase and sense riboprobe generated using T₇ polymerase. Radioactive in situ hybridizations were performed on paraformaldehyde-fixed, paraffin-embedded sections. β-galactosidase staining was described elsewhere (Saito et al., 2003, Am J Physiol Renal Physiol. 284: F1164-1170).

RNA isolation: Total RNA was extracted from tissues using TRIZOL (Invitrogen, CA) and RNeasy Mini-Kit (Qiagen). Reverse transcription was carried out on 400 ng of RNA using Taqman Reverse transcription reagents (Applied Biosystems). The resulting cDNA (40 ng) was used for quantitative real time PCR.

Quantitative real time PCR Analysis of Gene Expression: TaqMan gene expression assays (Applied Biosystems, Foster City, Calif.; catalog No. 4331182) for FP (Mm004360055_m1), RENIN (Mm02342889_g1), TNFα (Mm00443258_m1), IL-1β (Mm00434228_m1), IL-12β (Mm01351787_m1), IL-4 (Mm00445259_m1,) IL-6 (Mm00446190_m1), IL-18 (Mm00434225_m1), iNOS (Mm01309902_m1), TGFβ (Mm00441724_m1), INFγ (Mm00801778_m1), IL-10 (Mm00439616_m1) were performed on an ABI Prism 7900 Sequence Detection System. Results were normalized with 18S rRNA (Hs99999901_s1).

Statistical Analysis: Data are expressed as mean±SEM. Analyses were performed by initially by ANOVA and, if appropriate by subsequent pairwise comparisons. Distribution free approaches were utilized to avoid assumptions as to the normality of the distributions of the variables involved. Differences were considered statistically significant at P<0.05. Prism 4.0 software (GraphPad InStat 3) was used for all the calculations.

The experimental results are now described.

### Experimental Example 1

Using C57BL/6 mice and standard techniques in the art, mice having a knockout (KO) of the Ldl receptor (Ldlr), the prostaglandin F_{2α} (FP) receptor or both (double knockout; DKO) were generated. Knockout of Ldlr results in hyperlipidemic mice. Blood pressure was measured in wild type (WT) mice, Ldlr^{-/-} (LDLR KO) mice, FP^{-/-} (FPKO) mice, and Ldlr^{-/-}/FP^{-/-} mice using a tail cuff and/or telemetry.

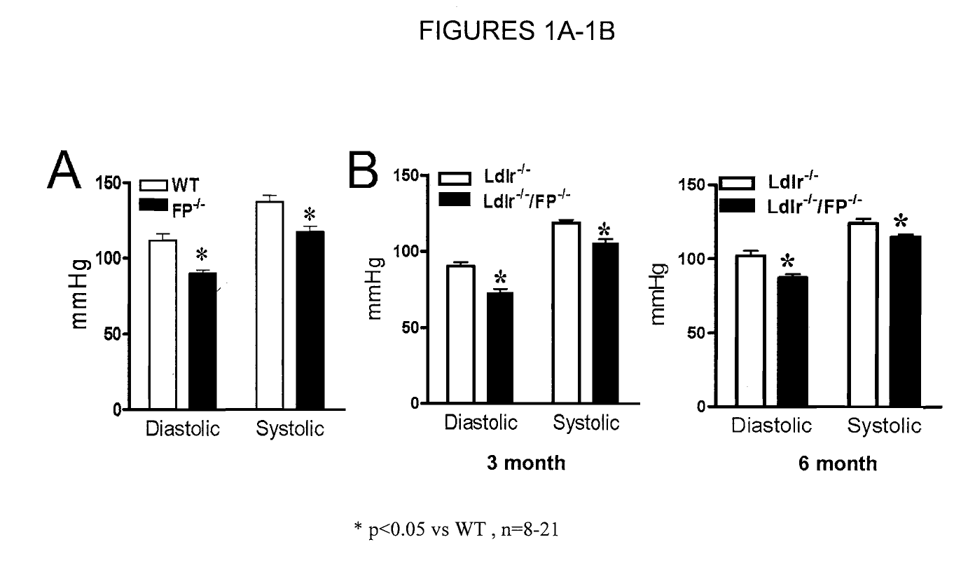

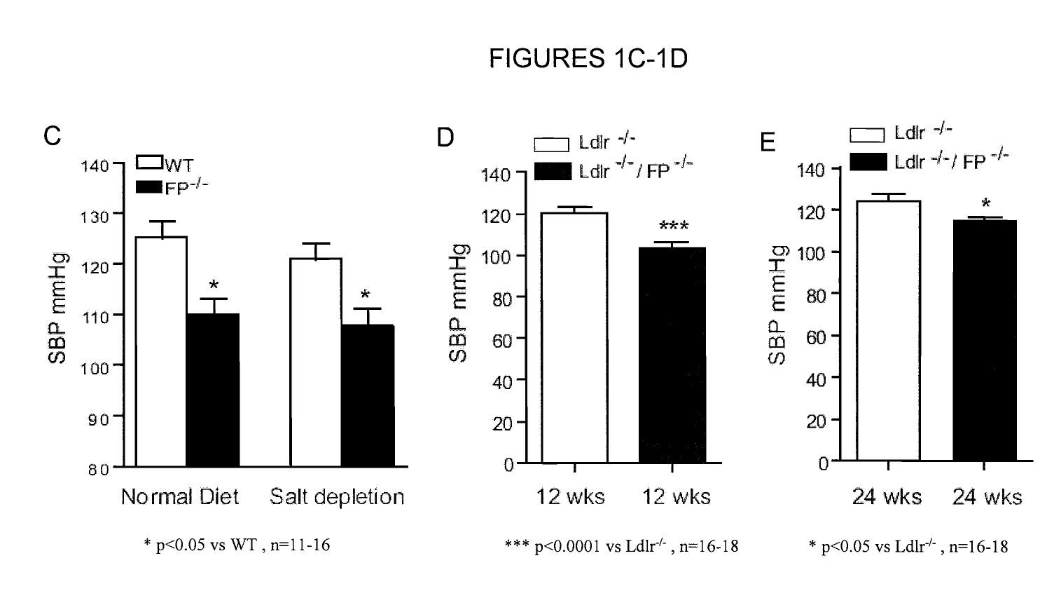

The deletion of FP reduced blood pressure significantly in both normolipidemic mice and in hyperlipidemic mice (FIGS. 1A and 1B). This result indicates that FP plays a role in maintaining blood pressure.

Resting systolic pressure was lower in normolipidemic FP^{-/-} mice (normal diet: 109.8±3.3 mmHg, n=11; salt depletion: 107.7±3.4 mmHg, n=11) compared with WT controls (normal diet: 125.3±3.2 mmHg, n=11, p<0.001; salt depletion: 121.0±3.3 mmHg, n=16; p<0.01) (FIG. 1C) and in hyperlipidemic Ldlr^{-/-}/FP^{-/-} mice (103.3±3.26, n=16, 12 weeks HFD; 115.2±1.53, n=16, 24 weeks HFD) compared with Ldlr^{-/-}/controls (120.1±3.16, n=16, p<0.0001, 12 weeks HFD; 124.2±3.18, n=16, p<0.05, 24 weeks HFD; FIGS. 1D and 1E). The difference in blood pressure between FP^{-/-} mice and controls was sustained throughout the 24 hr period in the telemetric studies. Tachycardia, reactive to hypotension, did not differ between the groups. Neither BP nor heart rate differed between normolipidemic and hyperlipidemic mice.

### Experimental Example 2

To assess the effect of FP knockout on the activation of the renin-angiotensin-aldosterone system, plasma renin activity (PRA), plasma angiotensin I (Ang I) and plasma aldosterone concentration (PAC) were measured using commercially-available kits in mice on normal diets and mice on a salt depleted diet. Salt depletion was induced by a low-salt diet (0.12% NaCL) plus intraperitoneal injection of furosemide (25 mg/kg) for one week. Salt depletion is known to activate the RAA system.

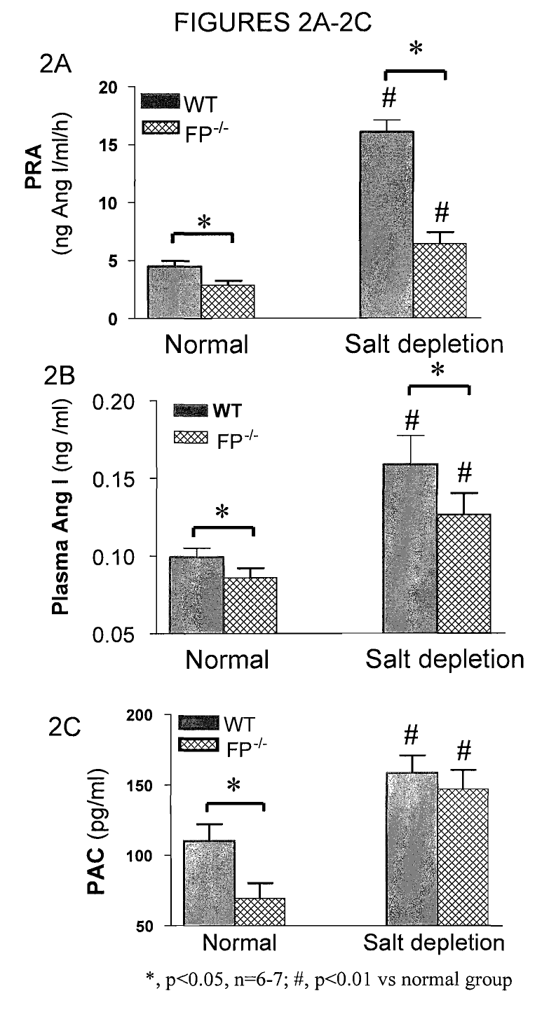

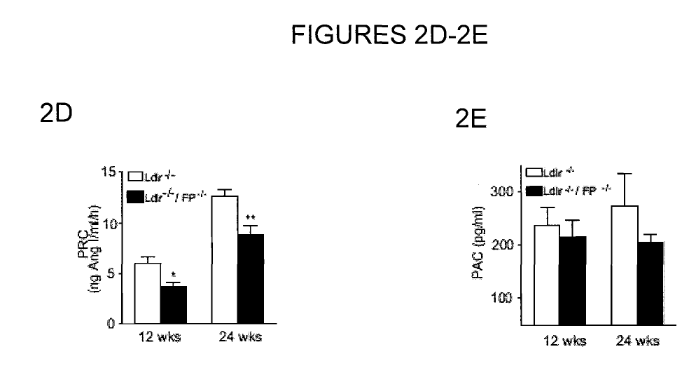

All three measures of activation of the RAA system were decreased in FP^{-/-} mice (FIGS. 2A-2C). Salt depletion activated the RAAS and exacerbated the impact of FP deletion on plasma renin concentration and on Ang I (FIGS. 2A and 2B), but not aldosterone secretion (FIG. 2C), suggesting that other angiotensin (AT) independent factors (such as adrenergic control), may be involved in the regulation of aldosterone secretion in response to salt depletion. Similar results were obtained in hyperlipidemic mice (FIGS. 2D and 2E). These data indicate that FP plays a role in activation of the RAA system.

### Experimental Example 3

Real-time reverse transcription PCR(RT-PCR) quantification of mRNA expression for the two isoforms of the AT1 antiotensin receptor, AT1a and AT1b, was performed. AT1 mediates many of the known physiological actions of angiotensin II (Ang II).

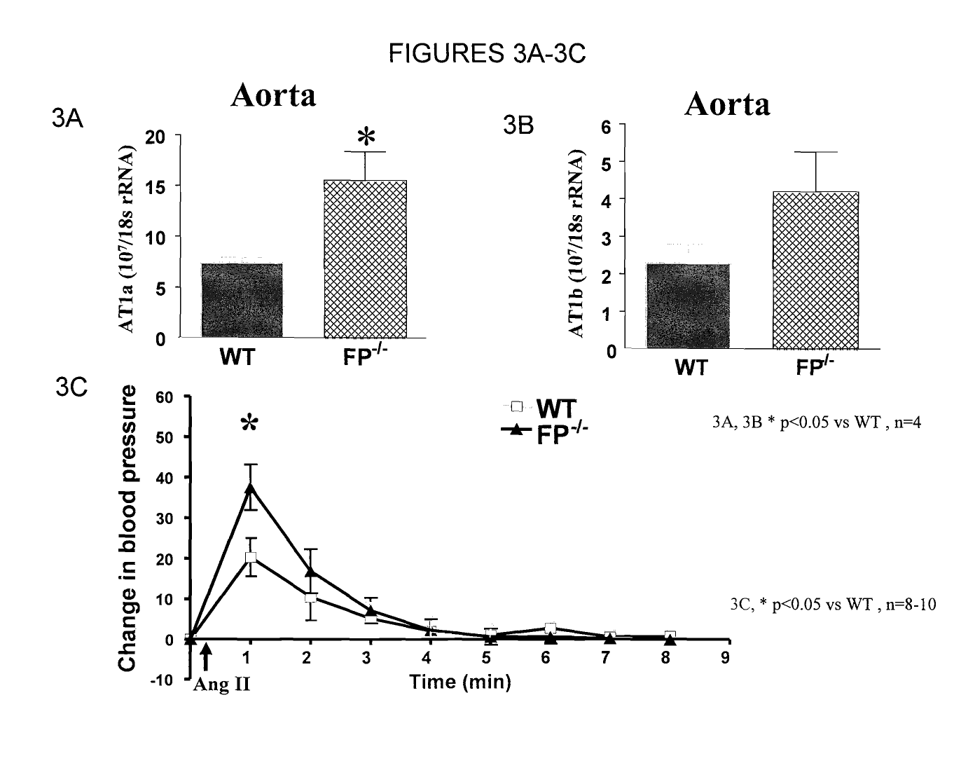

Deletion of FP resulted in increased expression of both aortic AT1a and aortic AT1b, compared to expression in WT mice (FIGS. 3A and 3B). mRNA expression of the dominant isoform, AT1a, increased in aorta in the FP^{-/-} mice (7.37±0.56×10⁷/18S rRNA vs 15.54±2.84×10⁷/18S rRNA, n=4, p<0.05, FIG. 3A) compared to WT controls. A less pronounced increase was observed in AT1b expression (p=0.13, FIG. 3B).

To examine the acute effect of Ang II on blood pressure, WT and FPKO mice were sedated and subjected to a bolus infusion of Ang II. Blood pressure was measured directly using an arterial catheter connected to a pressure transducer. After the equilibration period and the baseline BP was recorded, mice were injected with a bolus of Ang II.

The bolus of Ang II induced a greater increase in blood pressure in FP knockout mice compared to WT mice (FIG. 3C). Specifically, the hypertensive response to acute intracarotid infusion of Ang II peptide (50 ng/kg), was increased significantly in FP KO mice compared to WT controls (37.5±5.6 vs 20.3±4.7, n=7, p<0.05).

These data suggest that the decreased activation of the RAA system, resulting from deletion of FP, upregulates expression of Ang II receptors, possibly as a compensatory mechanism.

### Experimental Example 4

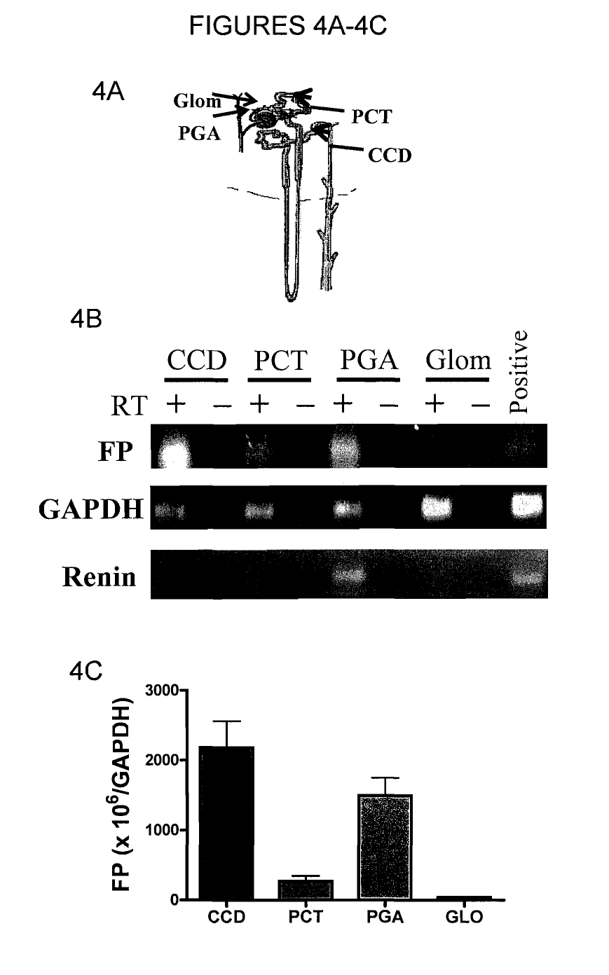

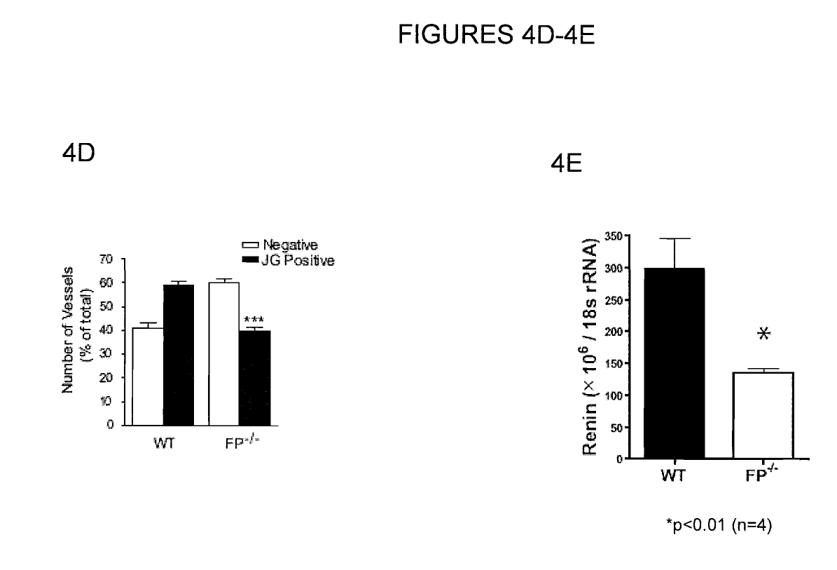

Consistent with a previous report, intense specific FP signals were detected using in situ hybridization with an FP antisense probe in epithelial tubules in the renal cortex (CCD) of WT mice; no detectable signal was seen with an FP sense probe. Expression of FP in four different parts of the kidney, proximal convoluted tubule (PCT), cortical collecting duct (CCD), glomerula (glom) and pre-glomerula artery (PGA), was assessed by real-time RT-PCR. FP was expressed in both the CCD and the PGA (FIG. 4A-4C).

Since decreased renin activity was observed in FP^{-/-} mice, the extension of granulation in afferent arterioles was quantified. The high-contrast regions seen at the end of afferent arterioles represent clusters of renin-positive cells as documented previously. The extent of visible granulation in the vascular trees at the terminal ends of the arterioles is decreased significantly in FP^{-/-} mice (40% renin positive, p<0.0001) compared to WT controls (59% renin positive; FIG. 4D). Kidney renin RNA expression was also downregulated significantly in FP^{-/-} mice (FIG. 4E).

Detection of FP receptors in proximal afferent glomerular arterioles and depression of the RAAS in FP Kos suggests that FP activation may mediate renin secretion in JG cells. To directly assess the effect of FP agonists on juxtaglomerular apparatus (JG) cells, JG cells were isolated from WT or FPKO mice and cultured in vitro. An prostanoid agonist was administered as different concentrations and the expression of renin was monitored using real time RT-PCR. Two FP agonists, latanoprost and travoprost, and an prostacyclin receptor (IP) agonist, cicaprost, were tested.

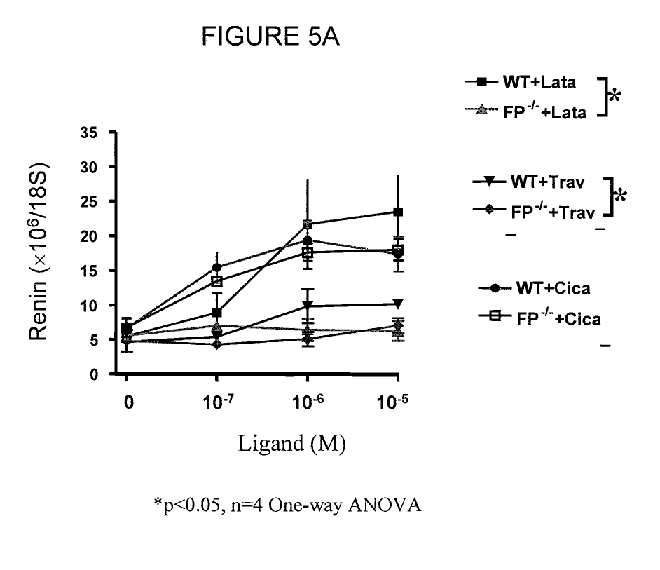

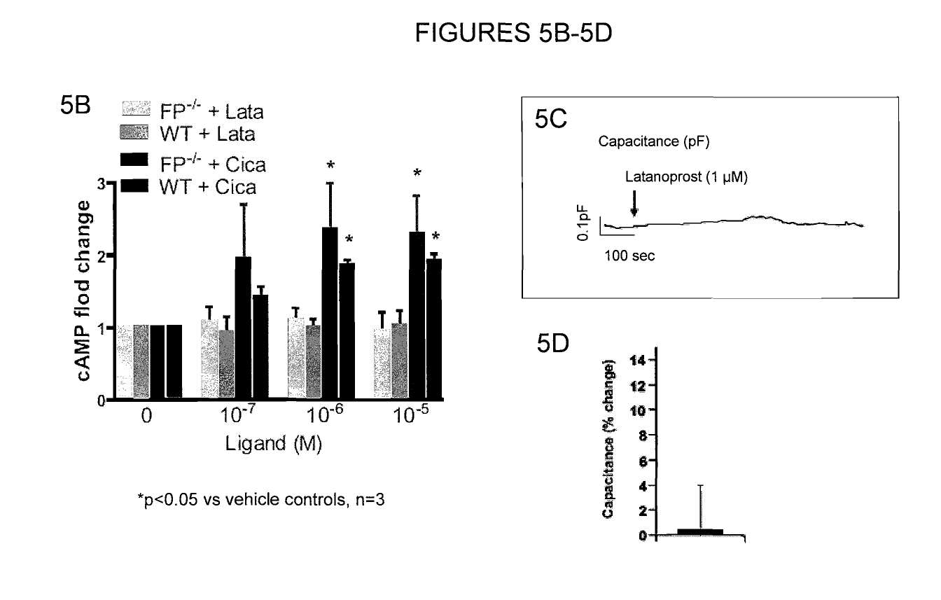

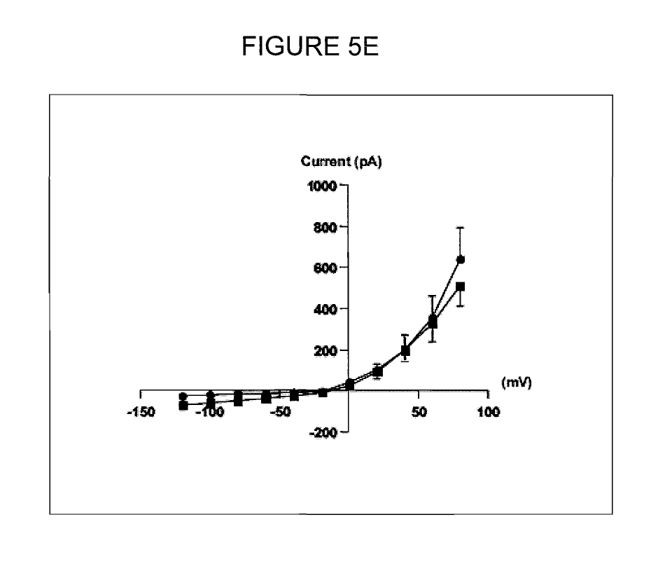

As shown in FIG. 5A, expression of renin in JG cells from WT mice was stimulated by all three agonists. Expression of renin in JG cells from FPKO mice was stimulated by cicaprost. Notably, expression of renin in JG cells from FPKO mice was not stimulated by either FP agonist. This data suggests that PGF_{2α} binding to PGF_{2α} receptors present on JG cells induces renin expression. By contrast, the effects of Cicaprost as a renin secretagogue was similar in WT and FP^{-/-} mice. However, despite the increase in JG renin, Latanoprost, unlike Cicaprost, failed to increase JG cell cAMP (FIG. 5B), a mediator of renin expression and exocytosis. Furthermore patch-clamp studies using single WT JG cells in vitro, incubated with Latanoprost, showed no change in membrane capacitance (FIG. 5C-5E), suggesting that the FP does not directly mediate renin exocytosis.

### Experimental Example 5

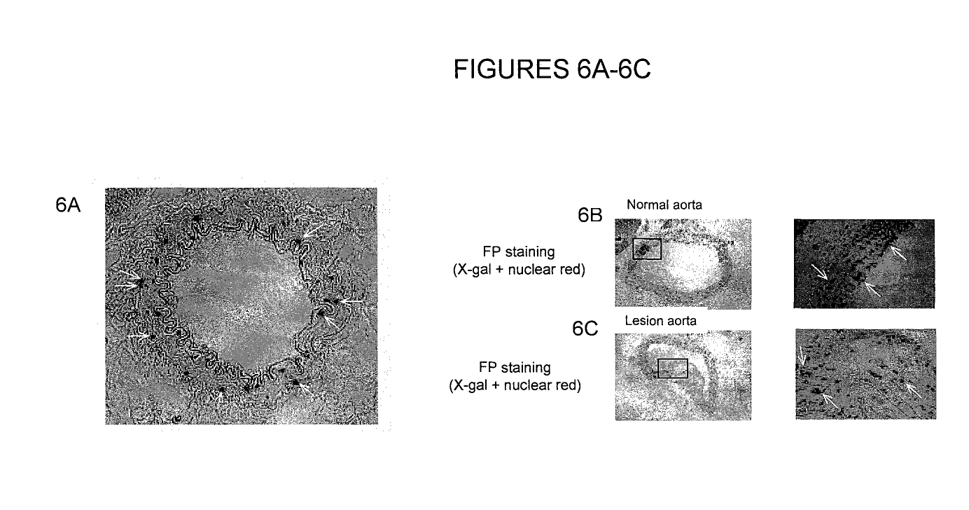

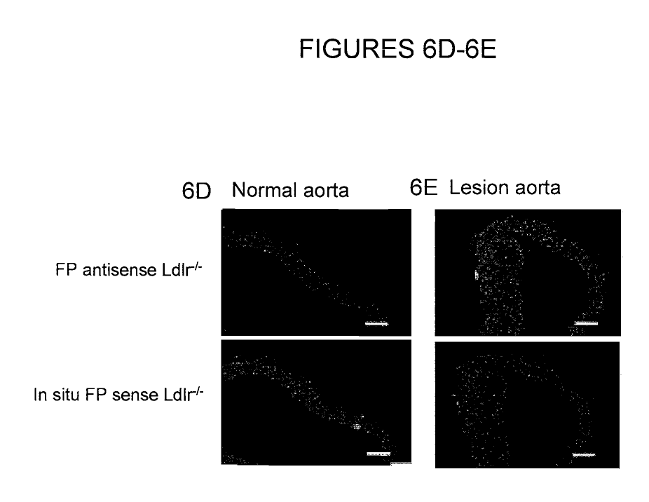

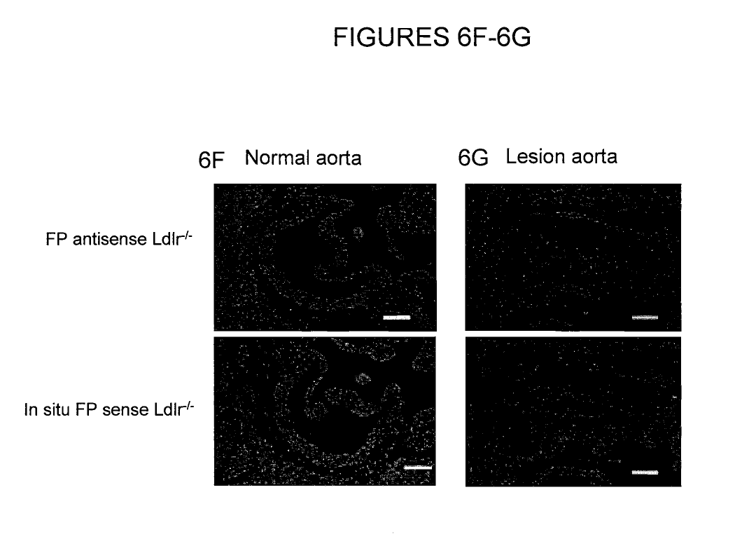

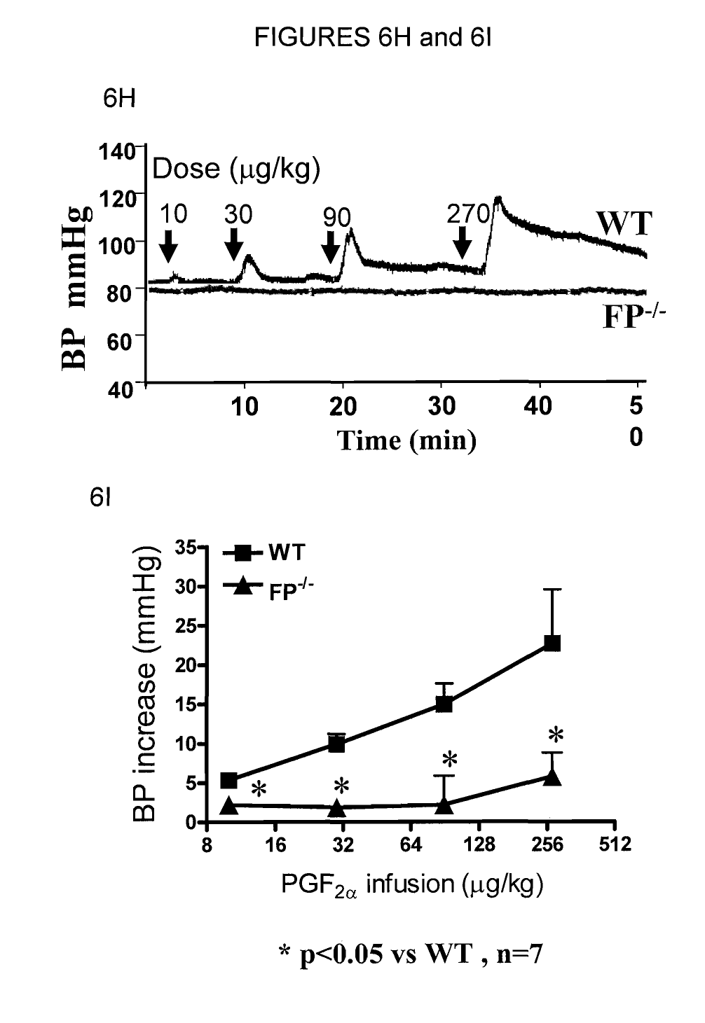

Given that the lacZ gene was knocked into the endogenous FP receptor locus in the FP targeting allele, the receptor expression pattern was monitored by mapping β-galactosidase (β-gal) activity. Scattered activity of β-gal was detectable in the medial smooth muscle layer of renal small arteries in FP^{+/-} mice (FIG. 6A), but was not evident in FP^{+/-} mouse aorta nor in the atherosclerotic lesions of Ldlr^{-/-}/FP^{-/-} mice (FIGS. 6B-6G). To assess the systemic effect of PGF_{2α}, WT and FPKO mice were sedated in order to measure blood pressure directly in the heart. PGF_{2α} was administered and blood pressure was monitored. Once blood pressure returned to baseline, the next dose of PGF_{2α} was administered (FIG. 6H). Four doses of PGF_{2α} were tested: 10 μg/kg body weight, 30 μg/kg, 90 μg/kg and 270 10 μg/kg.

Administration of PGF_{2α} increased blood pressure in WT mice but not FPKO mice (FIGS. 6H and 6I). The increase in WT mice was dose dependent (FIG. 6I). Thus, systemic pressor induced by PGF_{2α} is disrupted in FPKO mice. The rise in blood pressure at the highest rate of infusion in the FP^{-/-} mice likely reflects the weak affinity of PGF_{2α} for EP1 and/or EP3 receptors.

### Experimental Example 6

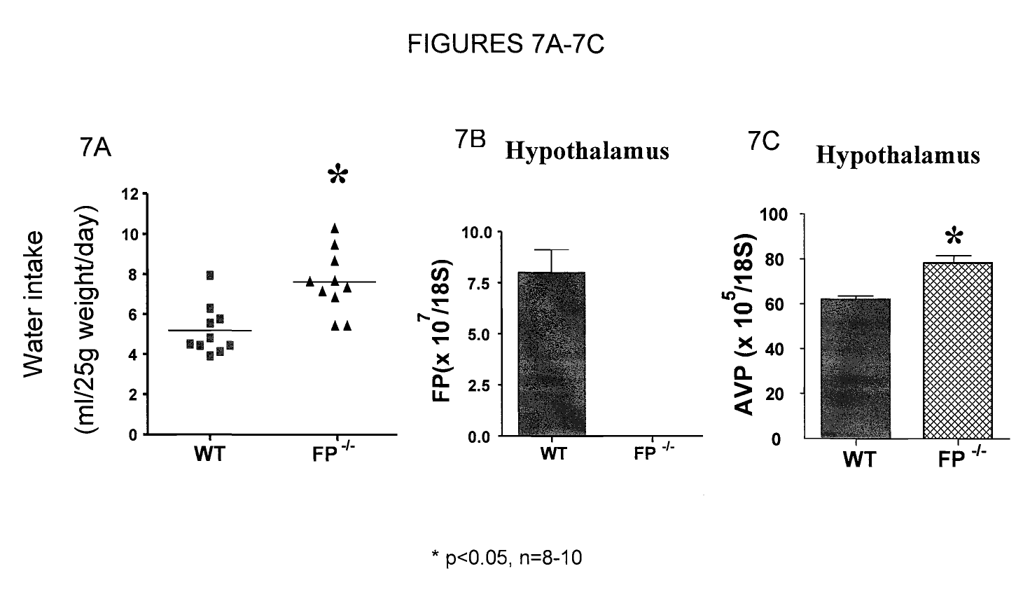

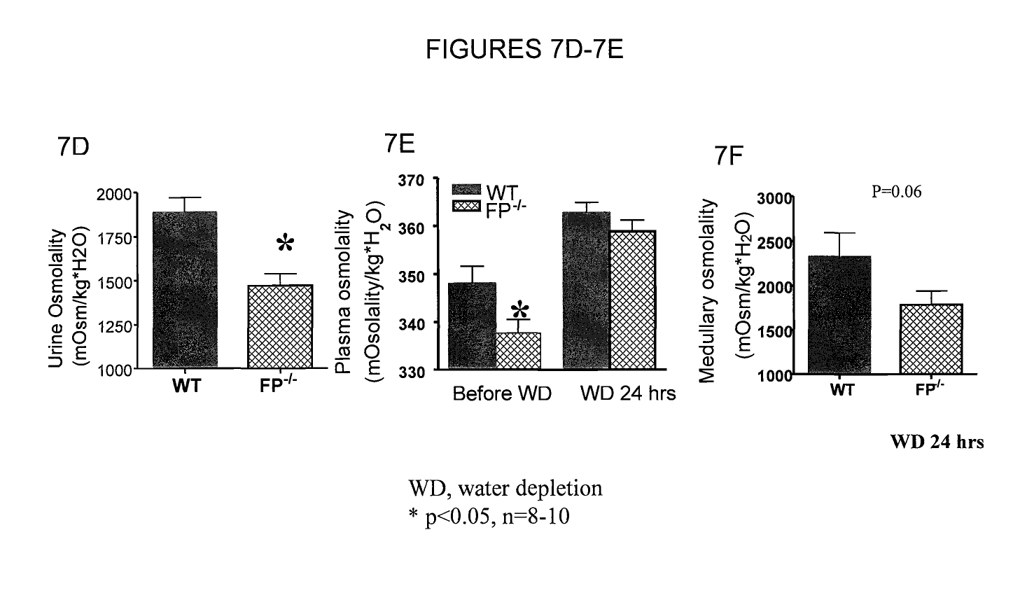

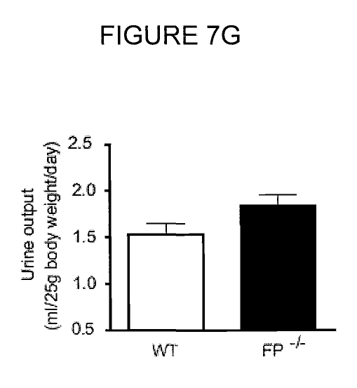

The effect of deletion of FP on water metabolism was examined. Both daily water consumption (WT, 5.181±0.39 ml/25 g body weight; FP^{-/-}, 7.621±0.49 ml/25 g body weight, n=10, P<0.05) and urine output (WT, 1.527±0.12 ml/25 g body weight; FP^{-/-}, 1.845±0.11 ml/25 g body weight, n=9, P=0.08) tended to be greater in FP^{-/-} mice than controls (FIGS. 7A and 7G, respectively). Moreover, both urinary (WT, 1888±82.40 mOsm/kg*H₂O vs FP^{-/-},1473±67.16 mOsm/kg*H₂O, n=6-9, P<0.05) and plasma (WT, 313.2±3.5 mOsm/kg*H₂O vs FP^{-/-}, 302.7±2.8 mOsm/kg*H₂O n=6, P<0.05) osmolality were reduced in FP^{-/-} mice (FIGS. 7D and 7E, respectively). Plasma osmolality rose significantly to a similar degree in FP^{-/-} and WT mice (FIG. 7E) that were subjected to 24 hr water deprivation. However, a mild defect in regulation of renal medullary osmolality was apparent (1859±84.3 mOsm/kg*H₂O in FP^{-/-} vs 2329±197.8 mOsm/kg*H₂O in WT; n=12-14, P<0.05; FIG. 7F). Activation of the FP inhibits water absorption in the rabbit collecting duct in vitro, implicating arginine-vasopressin (AVP). The FP was detected in microdissected WT hypothalamus (FIG. 7B), and hypothalamic AVP was upregulated in the FP^{-/-} mice (FIG. 7C). Without being bound by theory, it is thought that the increase in AVP by FP deletion, results in increased water uptake and consequently decrease in osmolality.

### Experimental Example 7

Ldlr KO mice are known to have increased atherogenesis compared to WT mice. To examine the effect of FP deletion on atherogenesis in hyperlipidemic mice, aortas (from the aortic root to the iliac bifurcation) were obtained from Ldlr KO and Ldlr/FP DKO mice and lipid deposition was assessed. The aorta was opened longitudinally from the aortic root to the iliac bifurcation, fixed and stained with oil red O. The extent of atherosclerosis was determined using the en face method (Morishita et al., 1990, J. Clin. Invest. 86: 1885-91).

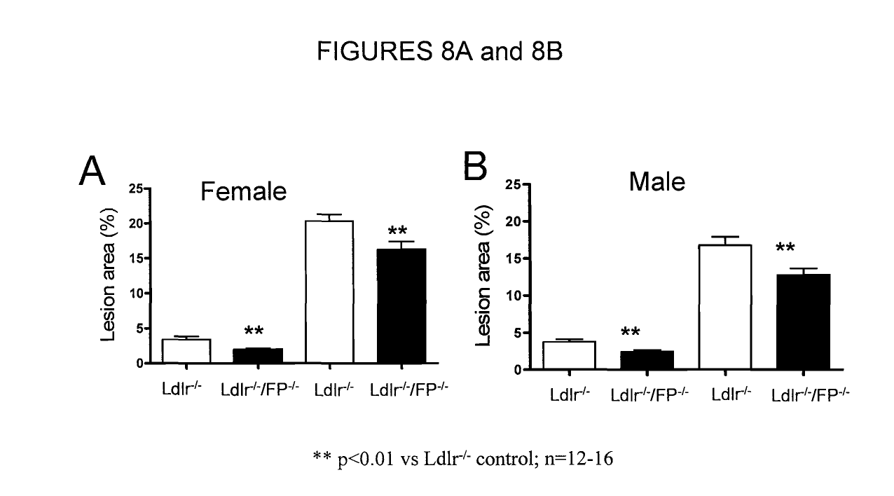

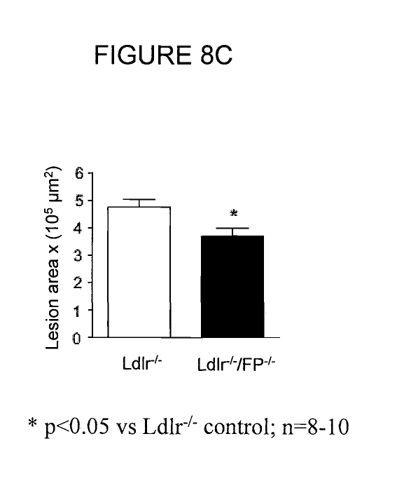

Total body weight and plasma cholesterol did not differ between Ldlr^{-/-} control and Ldlr^{-/-}/FP^{-/-} mice. Atherosclerotic lesional area, calculated en face was reduced by FP deletion (males, average 42% reduction; p=0.006 at 12 wks on a HFD; average 20% reduction; p=0.01 at 24 wks HFD); (females, average 37% reduction; p=0.003 at 12 wks HFD, 24% reduction; p=0.009 at 24 wks HFD) (FIGS. 8A and 8B). This result indicates the FP deletion retards atherogenesis.

Lesional burden, as assessed by cross-sectional analysis of the aortic root, was also reduced in Ldlr^{-/-}/FP^{-/-} mice (3.7±0.2×10⁵ μm² vs 4.7±0.2×10⁵ μm²; p=0.02) (FIG. 8C). Both in situ and X-gal staining of aortas and aortic root sections failed to detect FP expression in either normal or hyperlipidemic mice (FIGS. 6B-6G).

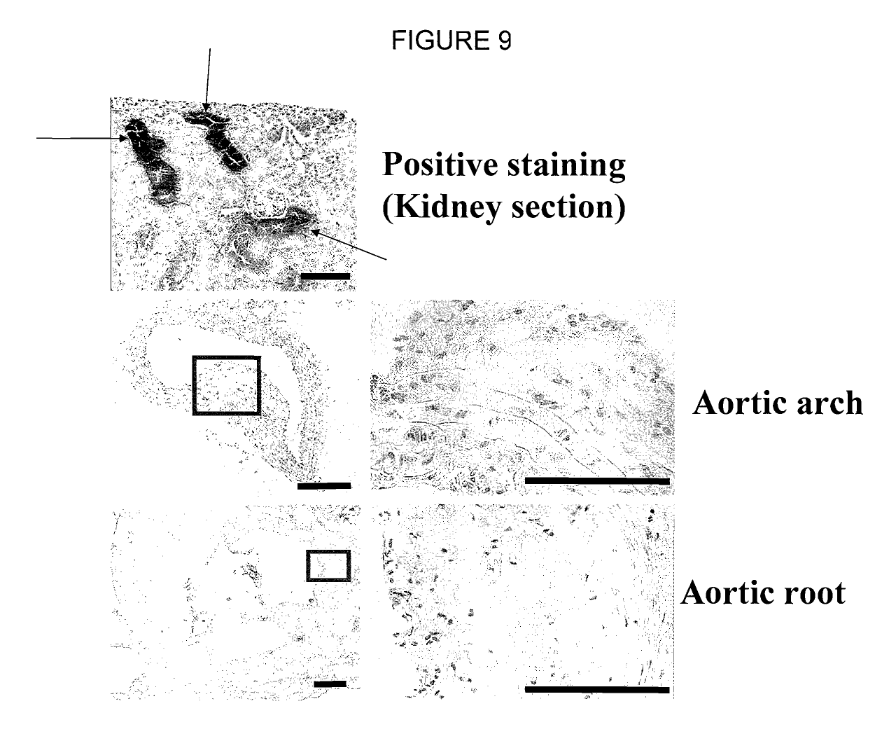

FPKO mice are transgenic mice having a LacZ gene functionally linked to FP promoter, with complete disruption of the FP gene. Expression of FP can therefore be detected by examining tissue sections from FPKO or Ldlr/FP DKO mice for LacZ expression. In Ldlr/FP DKO mice, no lacZ expression was detected in the aorta or athero lesions, indicating there is no expression of FP in the aorta or in the athero lesions (FIG. 9).

### Experimental Example 8

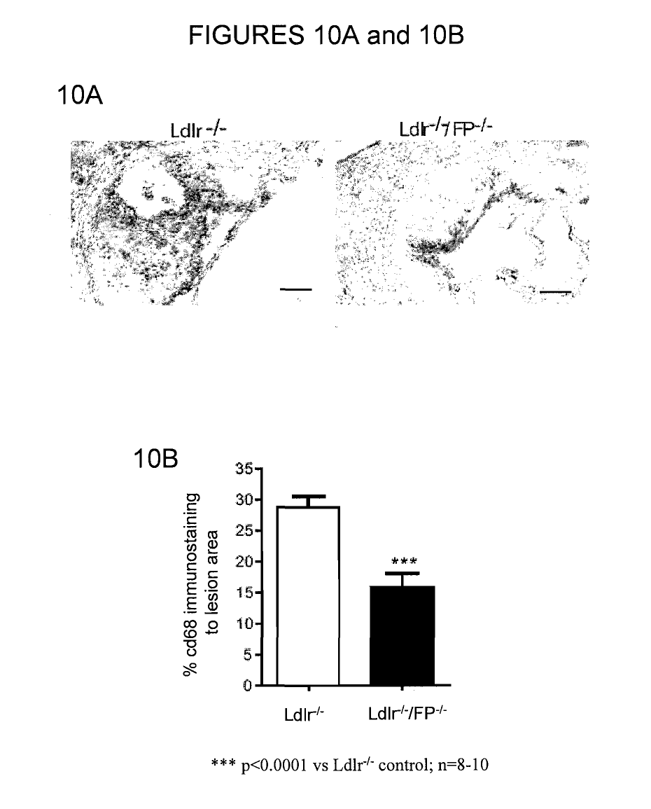

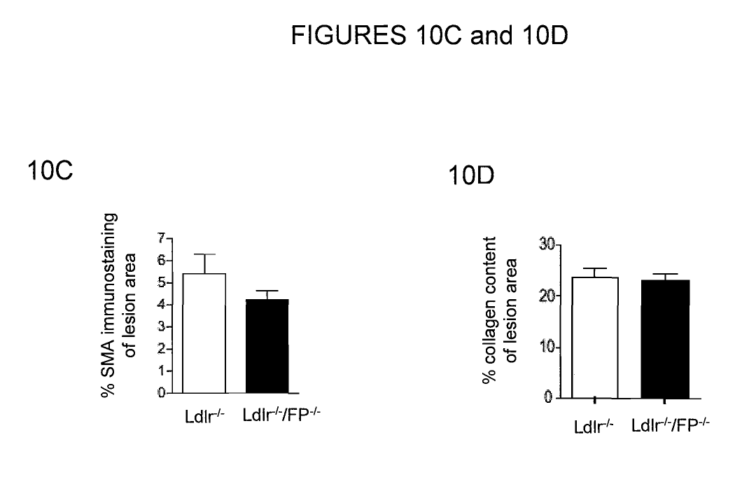

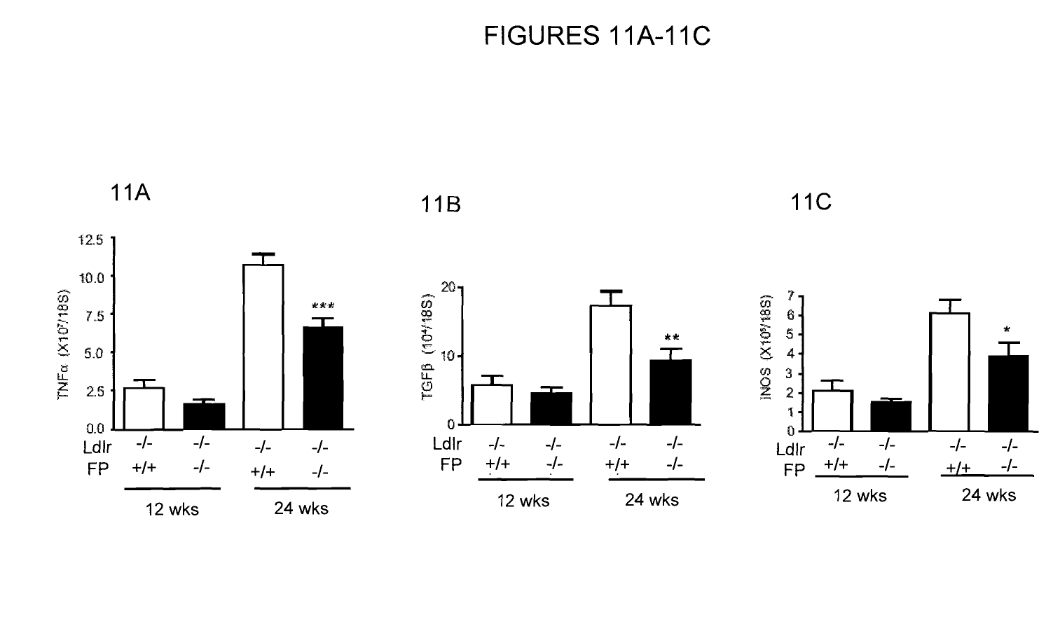

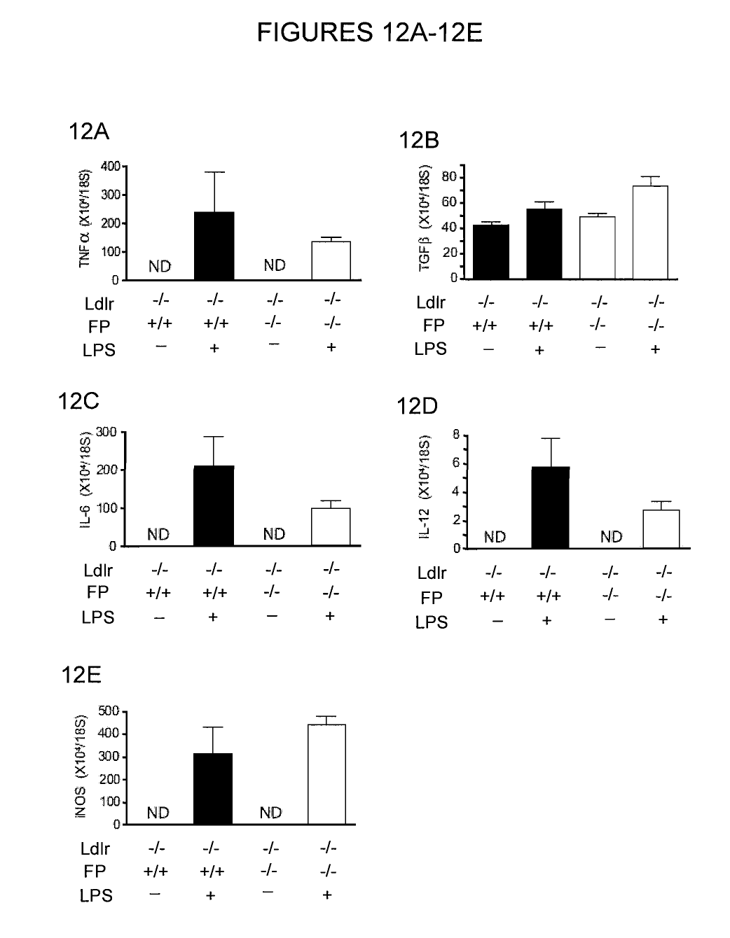

Surface macrophage staining (CD68) as a percentage of total lesional area at the aortic root (average 46%) was reduced (FIGS. 10A and 10B; n=8, p=0.0007) in Ldlr^{-/-}/FP^{-/-} mice. There was no observable difference in the immunostaining of the other cellular and matrix markers tested (Smooth muscle cell marker-α-actin, FIG. 10C; Endothelial cell marker-PECAM-1 (not shown); Collagen, FIG. 10D). Correspondingly, lesional expression of the inflammatory cytokines, TNF_{α}, TGF_{β} and inducible nitric oxide enzyme (iNOS) was reduced in Ldlr^{-/-}/FP^{-/-} mice at 24 weeks on the HFD (FIGS. 11A-11C). This raises the possibility that PGF_{2α} might regulate macrophage cytokine production directly and thereby influence atherogenesis. However, FP deletion did not alter basal or LPS stimulated cytokine production ex vivo (TNF_{α}, FIG. 12A; TGF_{β}, FIG. 12B; IL-6, FIG. 12C; IL-12, FIG. 12D; iNOS, FIG. 12E) in peritoneal macrophages from Ldlr^{-/-}, nor was FP expression detectable by real time RT-PCR in cultured peritoneal macrophages as previously reported (Rouzer et al., 2005, J Lipid Res. 46: 1027-1037). Thus, the reduction in lesional cytokines might result from, rather than mediate, the retardation of atherogenesis in Ldlr^{-/-}/FP^{-/-} mice.

As shown herein, deletion of the FP results in hypotension coincident with depression of the RAAS and occurs despite an augmented response to infused Ang II. Furthermore, despite renal tubular expression of the FP and a natriuretic response to PGF_{2α} infusion, urinary electrolyte excretion and the response to an aldosterone receptor antagonist is unaltered in FP^{-/-} mice. Thus, the mechanism by which deletion of this receptor reduces blood pressure is quite distinct from that observed with other prostanoids.

FP receptors were detected in the proximal afferent arteriole where JG cells are located, and FP agonists dose dependently induced renin expression in cultured JG cells. However, this did not coincide with an increase in JG cell cAMP, and patch-clamp studies using single JG cells treated with FP agonists, exhibited no change in membrane capacitance, suggesting that the FP does not directly mediate renin exocytosis. FP receptor expression is marked in afferent arterioles where renin granular cells were decreased in the FP^{-/-} mice. Thus, rather than acting directly, activation of the FP appears to regulate JG cell differentiation and consequent renin expression, explaining low RAAS activity in FP^{-/-} mice.

Hypertension facilitates atherogenesis, probably in part by causing endothelial dysfunction. While not wishing to be bound by theory, given that FP was in atherosclerotic (or normal) aorta, it appears likely that a reduction in blood pressure, consequent to disruption of the RAAS, or its consequences—for example secondary humoral adjustments—contributes to, if not explains the impact of FP deletion on atherogenesis. The mechanism by which atherogenesis is restrained is quite distinct: the FP is undetectable in either aortic smooth muscle cells or in lesional macrophages. Indeed, FP deletion fails to modulate macrophage cytokine release. Thus, while lesional macrophages are depleted and some inflammatory cytokines depressed in Ldlr^{-/-}/FP^{-/-} mice, this reflects, rather than causes, the reduction in plaque burden. Hence, restraint of atherogenesis in Ldlr^{-/-}/FP^{-/-} mice correlates with a reduction in systemic blood pressure that results from the impact of FP deletion on RAAS activation in the kidney.

In brief, the data presented demonstrate that deletion of the PGF_{2α} receptor results in a decreased activation of the RAA system, mediated by a decreased renin expression by JG cells. The decreased RAA system activation in turn induces an increase in expression of AT1 receptor. In addition, FP deletion induces upregulation of AVP expression in the hypothalamus with a increase in water uptake and a consequential decrease in urine, plasma and medullary osmolality. Furthermore, the hypotension caused by the deletion of FP coincides with and may be causative of a decrease in atherogenesis.

Genetic deletion of the PGF_{2α} receptor is comparable to complete inhibition of PGF_{2α} receptor activity. Thus, chemical inhibition of the PGF_{2α} receptor is expected to similarly reduce blood pressure and atherogenesis. Accordingly, identifying inhibitors of the PGF_{2α} receptor are expected to be candidate therapeutics for the treatment of hypertension and for decreasing the rate or extent of atherogenesis.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of treating hypertension in an individual, the method comprising administering a therapeutically effective amount of an inhibitor of prostaglandin F_{2α} receptor to the individual, wherein the inhibitor decreases blood pressure in the individual.

2. The method of claim 1, wherein said inhibitor of prostaglandin F_{2α} receptor is a selective inhibitor.

3. The method of claim 1, wherein said inhibitor of prostaglandin F_{2α} receptor is selected from the group consisting of AL-8810 (11 beta-fluoro-15-epi-15-indanyl-tetranor PGF_{2α}), PGF_{2α} dimethylamide, PGF_{2α} dimethylamine, and derivatives thereof.

4. The method of claim 1, wherein said hypertension is renin-dependent hypertension.

5. A method of alleviating atherogenesis in an individual, the method comprising administering a therapeutically effective amount of an inhibitor of prostaglandin F_{2α} receptor to the individual, wherein the inhibitor decreases blood pressure, thereby alleviating atherogenesis in the individual.

6. The method of claim 5, wherein said inhibitor of prostaglandin F_{2α} receptor is a selective inhibitor.

7. The method of claim 5, wherein said inhibitor of prostaglandin F_{2α} receptor is selected from the group consisting of AL-8810 (11 beta-fluoro-15-epi-15-indanyl-tetranor PGF_{2α}), PGF_{2α} dimethylamide, PGF_{2α} dimethylamine, and derivatives thereof.

8. A method of identifying a candidate therapeutic for the treatment of hypertension, the method comprising
identifying an inhibitor of prostaglandin F_{2α} receptor, thereby identifying a candidate therapeutic for the treatment of renin-dependent hypertension.

9. The method of claim 8, wherein the step of identifying an inhibitor of prostaglandin F_{2α} receptor comprises:
a. measuring a first level of prostaglandin F_{2α} receptor activity present in a cell in the presence of a prostaglandin F_{2α} receptor agonist;
b. administering a test compound to the cell;
c. measuring a second level of prostaglandin F_{2α} receptor activity in the cell,
wherein a test compound that reduces the second level of prostaglandin F_{2α} receptor activity compared to the first level of prostaglandin F_{2α} receptor activity is identified as a candidate therapeutic for the treatment of renin-dependent hypertension.

10. The method of claim 8, wherein the cell is a juxtaglomerular apparatus cell.

11. The method of claim 10, wherein the prostaglandin F_{2α} receptor activity is selected from the group consisting of: renin expression, renin release, plasma renin activity (PRA), plasma Ang II, plasma aldosterone concentration (PAC) and intracellular Ca²⁺.

12. The method of claim 10, wherein the juxtaglomerular apparatus cell is human.

13. The method of claim 10, wherein the cell is a non-human mammal juxtaglomerular apparatus cell that recombinantly expresses human prostaglandin F_{2α} receptor.

14. The method of claim 8, wherein the prostaglandin F_{2α} receptor is human.

15. The method of claim 8, wherein the hypertension is renin-dependent hypertension.

16. A method of identifying a candidate therapeutic for the treatment of hypertension, the method comprising
a. measuring a first level of prostaglandin F_{2α} receptor activity present in a non-human mammal that expresses prostaglandin F_{2α} receptor in juxtaglomerular cells in the presence of a prostaglandin F_{2α} receptor agonist;
b. administering a test compound to the non-human mammal;
c. measuring a second level of prostaglandin F_{2α} receptor activity in the non-human mammal,
wherein a test compound that reduces the second level of prostaglandin F_{2α} receptor activity compared to the first level of prostaglandin F_{2α} receptor activity is identified as a candidate therapeutic for the treatment of renin-dependent hypertension.

17. The method of claim 16, wherein the prostaglandin F_{2α} receptor activity is selected from the group consisting of: renin expression, renin release, plasma renin activity (PRA), plasma Ang II, plasma aldosterone concentration (PAC), blood pressure, AT1 expression, arginine vasopressin expression, and intracellular Ca²⁺.

18. The method of claim 16, wherein the non-human mammal recombinantly expresses human prostaglandin F_{2α} receptor.

19. The method of claim 18, wherein the recombinant human prostaglandin F_{2α} receptor is expressed in juxtaglomerular apparatus cells.

20. The method of claim 16, wherein said non-human mammal is transgenic for human prostaglandin F_{2α} receptor.

21. The method of claim 16, wherein the hypertension is renin-dependent hypertension.
